SEQUENCE LISTING (20 sequences)

SEQ ID NO: 1 - protein, 166 aa, Artificial
  chemically synthesized and optionally glycosylated
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 2 - protein, 165 aa, Artificial
  chemically synthesized, homogenously glycosylated
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

SEQ ID NO: 3 - protein, 166 aa, Artificial; identical to SEQ ID NO: 1
  chemically synthesized, homogenously glycosylated, modified

SEQ ID NO: 4 - protein, 166 aa, Artificial; identical to SEQ ID NO: 1
  chemically synthesized, homogenously glycosylated, modified

SEQ ID NO: 5 - protein, 88 aa, Artificial
  chemically synthesized, modified, glycosylated
CLLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 6 - protein, 50 aa, Artificial
  chemically synthesized, modified, glycosylated
CAEHCSLNENITVPDTKVNFYAWKRMEVGQQAVEVWQGLALLSEAVLRGQ

SEQ ID NO: 7 - protein, 28 aa, Artificial
  chemically synthesized, modified, glycosylated
APPRLICDSRVLERYLLEAKEAENITTG

SEQ ID NO: 8 - protein, 39 aa, Artificial
  chemically synthesized, modified
CPLRTITADTFRKLFRVYSNFLRGKLKLYTGEACRTGDR

SEQ ID NO: 9 - protein, 42 aa, Artificial
  chemically synthesized, modified, glycosylated
CSACPLRTITADTFRKLFRVYSNFLRGKLKLYTGEACRTGDR

SEQ ID NO: 10 - protein, 46 aa, Artificial
  chemically synthesized, modified, glycosylated
CLLVDSSQPWEPLQLHVDKAVSGLRSLTTLLRALGAQKEAISPPDA

SEQ ID NO: 11 - protein, 46 aa, Artificial
  chemically synthesized, modified, glycosylated
CLLVNSSQPWEPLQLHVDKAVSGLRSLTTLLRALGAQKEAISPPDA

SEQ ID NO: 12 - protein, 50 aa, Artificial; identical to SEQ ID NO: 6
  chemically synthesized, modified, glycosylated

SEQ ID NO: 13 - protein, 49 aa, Artificial
  chemically synthesized, modified, glycosylated
CEHCSLNENITVPDTKVNFYAWKRMEVGQQAVEVWQGLALLSEAVLRGQ

SEQ ID NO: 14 - protein, 29 aa, Artificial
  chemically synthesized, modified, glycosylated
APPRLICDSRVLERYLLEAKEAENITTGC

SEQ ID NO: 15 - protein, 88 aa, Artificial
  chemically synthesized, modified, glycosylated
CLLVNSSQPWEPLQLHVDKAVSGLRSLTTLLRALGAQKEAISPPDACSACPLRTITADTFRKLFRVYSNFLRGKLKLYTGEACRTGDR

SEQ ID NO: 16 - protein, 138 aa, Artificial
  chemically synthesized, modified, glycosylated
CAEHCSLNENITVPDTKVNFYAWKRMEVGQQAVEVWQGLALLSEAVLRGQCLLVNSSQPWEPLQLHVDKAVSGLRSLTTLLRALGAQKEAISPPDACSACPLRTITADTFRKLFRVYSNFLRGKLKLYTGEACRTGDR

SEQ ID NO: 17 - protein, 138 aa, Artificial
  chemically synthesized, modified, glycosylated
CAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 18 - protein, 38 aa, Artificial
  chemically synthesized, modified, glycosylated
CVEVWQGLALLSEAVLRGQALLVNSSQPWEPLQLHVDK

SEQ ID NO: 19 - protein, 166 aa, Artificial
  chemically synthesized, modified, glycosylated
APPRLICDSRVLERYLLEAKEAENITTGCCEHCSLNENITVPDTKVNFYAWKRMEVGQQAVEVWQGLALLSEAVLRGQCLLVNSSQPWEPLQLHVDKAVSGLRSLTTLLRALGAQKEAISPPDACSACPLRTITADTFRKLFRVYSNFLRGKLKLYTGEACRTGDR

SEQ ID NO: 20 - protein, 166 aa, Artificial; identical to SEQ ID NO: 1
  chemically synthesized, modified, glycosylated

(19) United States
(12) Patent Application Publication
(10) Pub. No.: US 2013/0323774 A1
(43) Pub. Date: Dec. 5, 2013

(54) HOMOGENOUS AND FULLY GLYCOSYLATED HUMAN ERYTHROPOIETIN

(71) Applicant: SLOAN-KETTERING INSTITUTE FOR CANCER RESEARCH, New York, NY (US)

(72) Inventors: Samuel J. Danishefsky, Englewood, NJ (US); Ping Wang, New York, NY (US); Suwei Dong, New York, NY (US); Malcolm Andrew Stephen Moore, New York, NY (US); Jae-hung Shieh, New York, NY (US); John Andrew Brailsford, New York, NY (US)

(73) Assignee: SLOAN-KETTERING INSTITUTE FOR CANCER RESEARCH, New York, NY (US)

(21) Appl. No.: 13/874,295

(22) Filed: Apr. 30, 2013

Related U.S. Application Data
(60) Provisional application No. 61/640,640, filed on Apr. 30, 2012.

(51) Int. Cl. C07K 14/505; G01N 33/74

(52) U.S. Cl. CPC C07K 14/505; G01N 33/746
USPC 435/29; 530/397; 530/322

(57) ABSTRACT

The present invention provides homogenous, fully-glycosylated, full length erythropoietin and the methods of producing the same.

### CROSS-REFERENCE TO RELATED APPLICATIONS

The present application claims priority to U.S. Provisional Application Ser. No. 61/640,640, filed Apr. 30, 2012, the entirety of which is incorporated herein by reference.

### GOVERNMENT SUPPORT

This invention was made with the support under the following government contract: CA28824, awarded by the National Institute of Health. The government has certain rights in the invention.

### BACKGROUND

Erythropoietin (EPO), a glycoprotein hormone secreted majorly by interstitial fibroblasts in the kidney, is encoded as a 166 amino acid polypeptide and found in nature as a 165-residue mature protein, which contains two disulfide bridges (Cys⁷-Cys¹⁶¹, Cys²⁹-Cys³³), three N-linked glycosylation sites (Asn²⁴, Asn³⁸, Asn⁸³), and one O-linked glycosylation site (Ser¹²⁶) ((a) Sytkowski, A. J. Erythropoietin; Wiley-VCH Verlag GmbH and Co. KGaA: Weinheim, 2004; (b) Jelkmann, W. Intern. Med. 2004, 43, 649-659). As the primary regulator of erythropoiesis, EPO elevates or maintains red-blood cell levels through a feedback mechanism involving the EPO receptor (EPOR) and the carbohydrate domains covalently attached to EPO ((a) J. C. Egrie, J. K. Browne, Nephrol. Dial. Transplant. 2001, 16 Suppl 3, 3-13; (b) T. Toyoda, T. Arakawa, H. Yamaguchi, J. Biochem. 2002, 131, 511-515; c) W. Jelkmann, Intern. Med. 2004, 43, 649-659). EPO has important physiological roles, and is used in treatment of anemia associated with renal failure and cancer chemotherapy. The role of glycosylation has been revealed to be extremely important for the in vitro and in vivo activities ((a) Higuchi, M.; Masayoshi, O.; Kuboniwa, H.; Tomonoh, K.; Shimonaka, Y.; Ochi, N. J. Biol. Chem. 1992, 267, 7703-7709; (b) Egrie, J. C.; Grant, J. R.; Gillies, D. K.; Aoki, K. H.; Strickland, T. W. Glycoconjugate J. 1993, 10, 263; (c) Egrie, J. C.; Browne, J. K. Br. J. Cancer 2001, 84 (51), 3-10), as well as for the stability of EPO (Narhi, L. O.; Arakawa, T.; Aoki, K. H.; Elmore, R.; Rohde, M. F.; Boone, T.; Strickland, T. W. J. Biol. Chem. 1991, 266, 23022-23026). The structure-function relationships of EPO glycoforms has not been well understood thus far, due to the heterogeneous nature of glycosylation in natural and recombinant EPO. Access to EPO as homogeneous glycoforms (homogeneously glycosylated EPO) with structurally well-defined glycans would be extremely valuable in the biological studies including the role of glycosylation ((a) M. R. Pratt, C. R. Bertozzi, Chem. Soc. Rev. 2005, 34, 58-68; (b) J. R. Rich, S. G. Withers, Nat. Chem. Biol. 2009, 5, 206-215; (c) D. P. Gamblin, E. M. Scanlan, B. G. Davis, Chem. Rev. 2009, 109, 131-163).

### SUMMARY

In some embodiments, the present invention provides a composition of homogeneously glycosylated erythropoietin. In some embodiments, the present invention provides a composition of homogeneous, fully glycosylated erythropoietin.

In some embodiments, the present invention provides methods for preparing a composition of homogenously glycosylated erythropoietin. In some embodiments, the present invention provides methods for preparing a composition of homogeneous, fully glycosylated erythropoietin. In some embodiments, the present invention provides methods for preparing a composition of homogeneous, fully glycosylated full-length erythropoietin. In some embodiments, the present invention provides methods for preparing a composition of homogenous, fully glycosylated full-length erythropoietin through chemical synthesis. In some embodiments, native chemical ligation and cysteine-free ligations based on a mild metal-free desulfurization protocol are employed in the chemical synthesis of homogenous fully glycosylated erythropoietin.

In some embodiments, the present invention provides methods to study the structure-function relationships of erythropoietin glycoforms using homogenously glycosylated erythropoietin. In some embodiments, the present invention provides methods to study the structure-function relationships of erythropoietin glycoforms using homogenous, fully glycosylated full-length erythropoietin.

### BRIEF DESCRIPTION OF THE DRAWINGS

FIG. 1. Effect of PROCRIT EPO and Synthetic EPO on Proliferation of Epo-dependent TF-1 erythroleukemic cells. 5,000 TF-1 cells/well/60 μl of IMDM medium containing 20% SR, 80 mM 2-mercaptoethanol, 2 mM L-glutamine, 50 units/ml penicillin, 50 μg/ml streptomycin in the presence or absence various doses of rhEPO or synthetic EPO was set up in a 384-wells plate in triplicates. After 72 hours culturing in a 5% CO₂ and humidified incubator, 6 μl of Alarma Blue (Invitrogen Inc. Grand Island, N.Y.) was added to each well and the cultures were incubated overnight. Fluorescence intensity of the culture in the 384-wells was measured using a Synergy H1 plate reader (BioTek).

FIG. 2. HPLC (a) and MS (b) for glycopeptide 4.

FIG. 3. HPLC (a) and MS (b) for glycopeptide 6.

FIG. 4. HPLC (a) and MS (b) for glycopeptide 7.

FIG. 5. HPLC (a) and MS (b) for glycopeptide 8.

FIG. 6. HPLC (a) and MS (b) for glycopeptide 9.

FIG. 7. HPLC (a) and MS (b) for glycopeptide 14.

FIG. 8. HPLC (a) and MS (b) for glycopeptide 23.

FIG. 9. HPLC (a) and MS (b) for glycopeptide 1.

FIG. 10. CD spectrum of fully synthetic, homogeneously glycosylated erythropoietin (chitobiose moieties at Asn²⁴, Asn³⁸ and Asn⁸³; and glycophorin at Ser¹²⁶).

### DETAILED DESCRIPTION OF CERTAIN EMBODIMENTS

### 1. Definitions

As used herein, the singular forms “a”, “an”, and “the” include the plural reference unless the context clearly indicates otherwise. Thus, for example, a reference to “a peptide” includes a plurality of such peptides.

The abbreviations as used herein corresponding to units of measure include: “g” means gram(s), “mg” means milligram(s), “ng” means nanogram(s), “kDa” means kilodalton(s), “° C.” means degree(s) Celsius, “min” means minute(s), “h” means hour(s), “1” means liter(s), “ml” means milliliter(s), “μl” means microliter(s), “M” means molar, “mM” means millimolar, “mmole” means millimole(s), and “RT” means room temperature. The abbreviations for chemical terms as used herein have the following definitions: “A” means alanine; “Ac” means acetyl; “AIBN” means 2,2′-azobis(2-methylpropionitrile); “Ala” means alanine; “Arg” means arginine; “Asn” means asparagine; “Asp” means aspartic acid; “Bn” means benzyl; “Boc” means tert-butyloxycarbonyl; “Bu” means butyl; “Bz” means benzoyl; “CAN” means ceric ammonium nitrate; “C-terminus” means carboxy terminus of a peptide or protein; “Cys” means cysteine' “D” means aspartic acid; “DIEA” means N,N-diisopropylethylamine; “DMAP” means N,N-dimethylaminopyridine; “DMF” means dimethyl formamide; “DMSO” means dimethyl sulfoxide; “DTBMP” means di-tert-butylmethylpyridine; “DTBP” means di-tert-butylpyridine; “Et” means ethyl; “Fmoc” means 9-fluorenylmethyloxycarbonyl; “Fuc” means L-Fucose; “G” means glycine; “Gal” means D-galactose; “GalNAc” means N-acetyl-D-galactosamine; “Glc” means D-glucose; “GlcNAc” means N-acetyl-D-glucosamine; “Gln” means glutamine; “Glu” means glutamic acid; “Gly” means glycine; “H” means histidine; “HATU” means 7-azahydroxybenzotriazolyl tetramethyluronium hexafluorophosphate; “His” means histidine; “Ile” means isoleucine; “K” means lysine; “KLH” means keyhole limpet hemocyanin; “L” means leucine; “Leu:” means leucine; “Lys” means lysine; “Man” means D-mannose; “MES-Na” means 2-mercaptoethanesulfonic acid, sodium salt; “N” means asparagine; “NAc” means N-acetyl; “NCL” means native chemical ligation; “Neu5Ac” means N-acetylneuraminic acid; “N-terminus” means amino-terminus of a peptide or protein; “O-linked” means linked through an ethereal oxygen; “PamCys” or “Pam3Cys” means tripalmitoyl-S-glycerylcysteinylserine; “PBS” means phosphate-buffered saline; “Ph” means phenyl; “PMB” means p-methoxybenzyl; “Pro” means proline; “PSA” means prostate specific antigen; “Py” means pyridine; “QS21” means a glycosteroidal immunoadjuvant; “R” means arginine; “S” means serine;“sat. aq.” means saturated aqueous; “Ser” means serine; “T” means threonine; “TBAF” means tetra-n-butylammonium fluoride; “TBS” means tert-butyldimethylsilyl; “tBu” means tert-butyl; “TCEP” means tricarboxyethylphosphine; “Tf” means trifluoromethanesulfonate; “TFA” means trifluoroacetic acid; “THF” means tetrahydrofuran; “Thr” means threonine; “Trp” means tryptophan; “V” means valine; “Val” means valine; and “W” means tryptophan.

Certain specific functional groups defined in the inventive method are described in more detail below. For purposes of this invention, the chemical elements are identified in accordance with the Periodic Table of the Elements, CAS version, Handbook of Chemistry and Physics, 75^{th} Ed., inside cover, and specific functional groups are defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

It will be appreciated that additional examples of generally applicable substituents are illustrated by the specific embodiments shown in the Examples which are described herein, but are not limited to these Examples.

By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “homogenously glycosylated erythropoietin” or “homogenous erythropoietin” refers to a composition of erythropoietin glycopeptides of which each molecule has the same glycosylation pattern, which means that: 1) each molecule of erythropoietin is glycosylated at the same glycosylation site(s); and 2) for a given glycosylation site, each molecule of erythropoietin has the same glycan. It will be appreciated that the terms “composition of homogeneously glycosylated erythropoietin” and “homogeneously glycosylated erythropoietin” are used interchangeably herein. The glycans at different glycosylation sites can be either the same or different. For example, for a homogenously glycosylated erythropoietin at Asn²⁴, Asn³⁸, Asn⁸³ and Ser¹²⁶, each molecule of erythropoietin: 1) is glycosylated at Asn²⁴, Asn³⁸, Asn⁸³ and Ser¹²⁶; and 2) has the same glycan at Asn²⁴, the same glycan at Asn³⁸, the same glycan at Asn⁸³, the same glycan at Ser¹²⁶, and the glycans at Asn²⁴, Asn³⁸, Asn⁸³ and Ser¹²⁶ can be the same or different on an individual molecule. An example of homogenously glycosylated erythropoietin is depicted below (Compound 3):

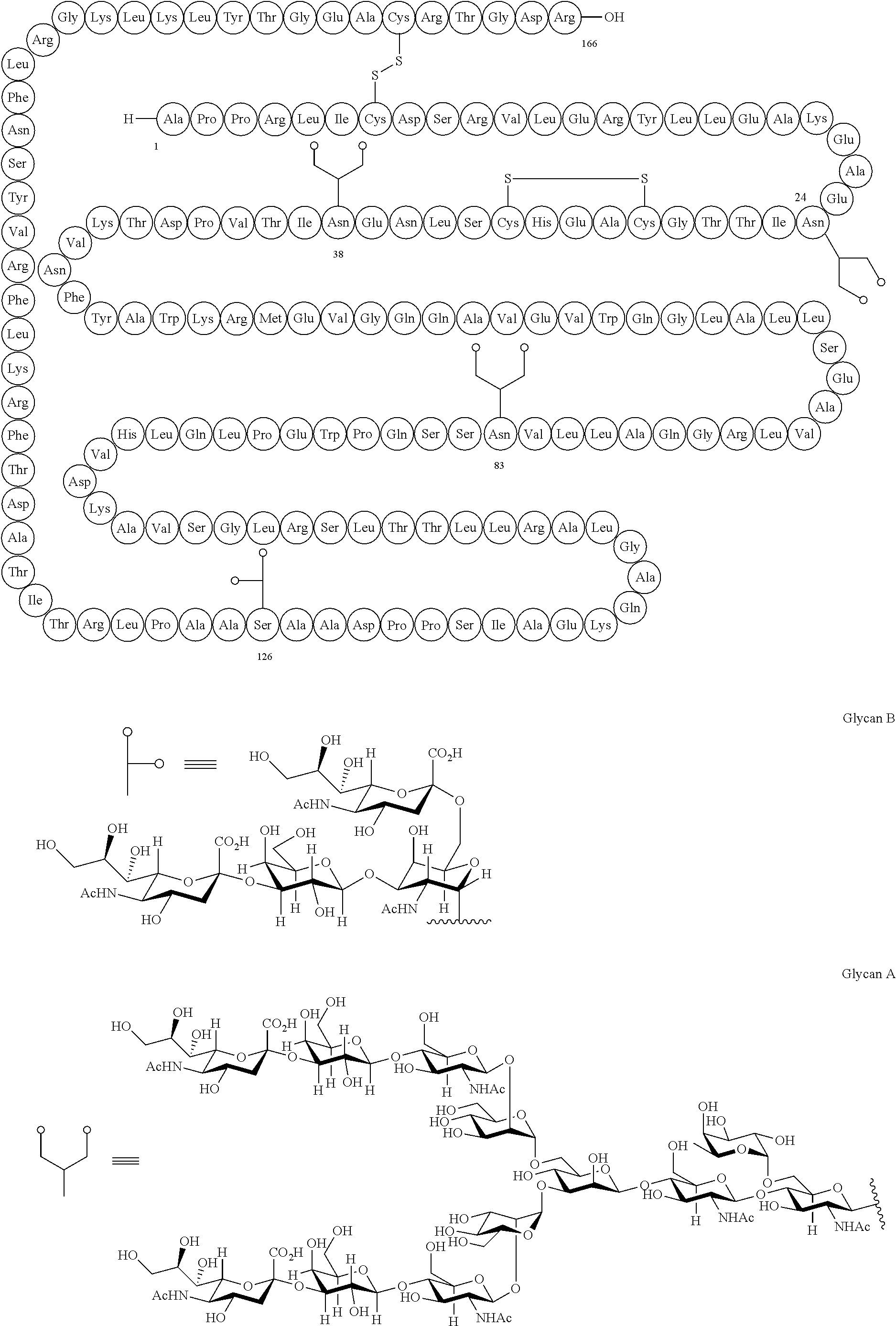

In this example, each erythropoietin molecule is glycosylated at Asn²⁴, Asn³⁸, Asn⁸³ and Ser¹²⁶, and each erythropoietin molecule has glycan A at Asn²⁴, glycan A at Asn³⁸, glycan A at Asn⁸³ and glycan B at Ser¹²⁶.

In some embodiments, “fully-glycosylated” refers to glycosylation of erythropoietin at three N-linked glycosylation sites (Asn²⁴, Asn³⁸, Asn⁸³) and one O-linked glycosylation site (Ser¹²⁶).

In some embodiments, “full-length erythropoietin” refers to erythropoietin that has 166 amino acid residues. In some embodiments, the primary amino acid sequence of erythropoietin is as follows:

Ala-Pro-Pro-Arg-Leu-Ile-Cys-Asp-Ser-Arg-Val-Leu-Glu-Arg-Tyr-Leu-Leu-Glu-Ala-Lys-
Glu-Ala-Glu-Asn-Ile-Thr-Thr-Gly-Cys-Ala-Glu-His-Cys-Ser-Leu-Asn-Glu-Asn-Ile-Thr-
Val-Pro-Asp-Thr-Lys-Val-Asn-Phe-Tyr-Ala-Trp-Lys-Arg-Met-Glu-Val-Gly-Gln-Gln-Ala-
Val-Glu-Val-Trp-Gln-Gly-Leu-Ala-Leu-Leu-Ser-Glu-Ala-Val-Leu-Arg-Gly-Gln-Ala-Leu-
Leu-Val-Asn-Ser-Ser-Gln-Pro-Trp-Glu-Pro-Leu-Gln-Leu-His-Val-Asp-Lys-Ala-Val-Ser-
Gly-Leu-Arg-Ser-Leu-Thr-Thr-Leu-Leu-Arg-Ala-Leu-Gly-Ala-Gln-Lys-Glu-Ala-Ile-Ser-
Pro-Pro-Asp-Ala-Ala-Ser-Ala-Ala-Pro-Leu-Arg-Thr-Ile-Thr-Ala-Asp-Thr-Phe-Arg-Lys-
Leu-Phe-Arg-Val-Tyr-Ser-Asn-Phe-Leu-Arg-Gly-Lys-Leu-Lys-Leu-Tyr-Thr-Gly-Glu-Ala-
Cys-Arg-Thr-Gly-Asp-Arg.

### 2. Description of Certain Embodiments of the Invention

In some embodiments, the present invention provides homogeneously glycosylated erythropoietin. In some embodiments, the present invention provides homogeneously glycosylated full-length erythropoietin. In some embodiments, the present invention provides homogeneous, fully-glycosylated full-length erythropoietin.

In some embodiments, the present invention provides homogeneous, fully glycosylated erythropoietin. In some embodiments, the present invention provides homogeneous, fully glycosylated erythropoietin glycosylated at Asn²⁴, Asn³⁸, Asn⁸³ and Ser¹²⁶.

In some embodiments, the present invention provides homogenous, fully glycosylated full-length erythropoietin. In some embodiments, the present invention provides homogeneous, fully glycosylated full-length erythropoietin, wherein the primary amino acid sequence of erythropoietin is as follows:

(SEQ ID NO: 1)
Ala-Pro-Pro-Arg-Leu-Ile-Cys-Asp-Ser-Arg-Val-Leu-Glu-Arg-Tyr-Leu-Leu-Glu-Ala-Lys-
Glu-Ala-Glu-Asn-Ile-Thr-Thr-Gly-Cys-Ala-Glu-His-Cys-Ser-Leu-Asn-Glu-Asn-Ile-Thr-
Val-Pro-Asp-Thr-Lys-Val-Asn-Phe-Tyr-Ala-Trp-Lys-Arg-Met-Glu-Val-Gly-Gln-Gln-Ala-
Val-Glu-Val-Trp-Gln-Gly-Leu-Ala-Leu-Leu-Ser-Glu-Ala-Val-Leu-Arg-Gly-Gln-Ala-Leu-
Leu-Val-Asn-Ser-Ser-Gln-Pro-Trp-Glu-Pro-Leu-Gln-Leu-His-Val-Asp-Lys-Ala-Val-Ser-
Gly-Leu-Arg-Ser-Leu-Thr-Thr-Leu-Leu-Arg-Ala-Leu-Gly-Ala-Gln-Lys-Glu-Ala-Ile-Ser-
Pro-Pro-Asp-Ala-Ala-Ser-Ala-Ala-Pro-Leu-Arg-Thr-Ile-Thr-Ala-Asp-Thr-Phe-Arg-Lys-
Leu-Phe-Arg-Val-Tyr-Ser-Asn-Phe-Leu-Arg-Gly-Lys-Leu-Lys-Leu-Tyr-Thr-Gly-Glu-Ala-
Cys-Arg-Thr-Gly-Asp-Arg;

and wherein the glycosylation sites are Asn²⁴, Asn³⁸, Asn⁸³ and Ser¹²⁶. In some embodiments, the fully glycosylated erythropoietin has an amino acid sequence as found in the natural mature erythropoietin. In some embodiments, the fully glycosylated erythropoietin has the primary amino acid sequence:

(SEQ ID NO: 2)
Ala-Pro-Pro-Arg-Leu-Ile-Cys-Asp-Ser-Arg-Val-Leu-Glu-Arg-Tyr-Leu-Leu-Glu-Ala-Lys-
Glu-Ala-Glu-Asn-Ile-Thr-Thr-Gly-Cys-Ala-Glu-His-Cys-Ser-Leu-Asn-Glu-Asn-Ile-Thr-
Val-Pro-Asp-Thr-Lys-Val-Asn-Phe-Tyr-Ala-Trp-Lys-Arg-Met-Glu-Val-Gly-Gln-Gln-Ala-
Val-Glu-Val-Trp-Gln-Gly-Leu-Ala-Leu-Leu-Ser-Glu-Ala-Val-Leu-Arg-Gly-Gln-Ala-Leu-
Leu-Val-Asn-Ser-Ser-Gln-Pro-Trp-Glu-Pro-Leu-Gln-Leu-His-Val-Asp-Lys-Ala-Val-Ser-
Gly-Leu-Arg-Ser-Leu-Thr-Thr-Leu-Leu-Arg-Ala-Leu-Gly-Ala-Gln-Lys-Glu-Ala-Ile-Ser-
Pro-Pro-Asp-Ala-Ala-Ser-Ala-Ala-Pro-Leu-Arg-Thr-Ile-Thr-Ala-Asp-Thr-Phe-Arg-Lys-
Leu-Phe-Arg-Val-Tyr-Ser-Asn-Phe-Leu-Arg-Gly-Lys-Leu-Lys-Leu-Tyr-Thr-Gly-Glu-Ala-
Cys-Arg-Thr-Gly-Asp,
wherein the glycosylation sites are Asn²⁴, Asn³⁸, Asn⁸³ and Ser¹²⁶.

In some embodiments, the homogenous, fully-glycosylated erythropoietin has one or more disulfide bonds. In some embodiments, the homogenous, fully-glycosylated erythropoietin has one disulfide bond. In some embodiments, the homogenous, fully-glycosylated erythropoietin has one disulfide bond formed between Cys⁷ and Cys¹⁶¹. In some embodiments, the homogenous, fully-glycosylated erythropoietin has one disulfide bond formed between Cys²⁹ and Cys³³. In some embodiments, the homogenous, fully-glycosylated erythropoietin has more than one disulfide bonds. In some embodiments, the homogenous, fully-glycosylated erythropoietin has two disulfide bonds. In some embodiments, the homogenous, fully-glycosylated erythropoietin has two disulfide bonds, one formed between Cys⁷ and Cys¹⁶¹, and the other Cys²⁹ and Cys³³.

In some embodiments, the homogeneous, fully-glycosylated erythropoietin is folded. In some embodiments, the homogeneous, fully-glycosylated erythropoietin is folded as found in nature. In some embodiments, the homogeneous, fully-glycosylated erythropoietin forms secondary structure. In some embodiments, the homogeneous, fully-glycosylated erythropoietin forms secondary structure as found in nature. In some embodiments, the homogeneous, fully-glycosylated erythropoietin forms tertiary structure. In some embodiments, the homogeneous, fully-glycosylated erythropoietin forms tertiary structure as fold in nature. In some embodiments, the homogeneous, fully-glycosylated erythropoietin forms quaternary structure. In some embodiments, the homogeneous, fully-glycosylated erythropoietin forms quaternary structure as found in nature. The secondary, tertiary and quaternary structures can be characterized by chemical, biochemical and structural biology means including, but not limited to chromatography, mass spectrometry, X-ray crystallography, NMR spectroscopy, and dual polarisation interferometry.

In some embodiments, each of the glycosylation sites of the homogeneous, fully glycosylated erythropoietin has a glycan independently selected from:

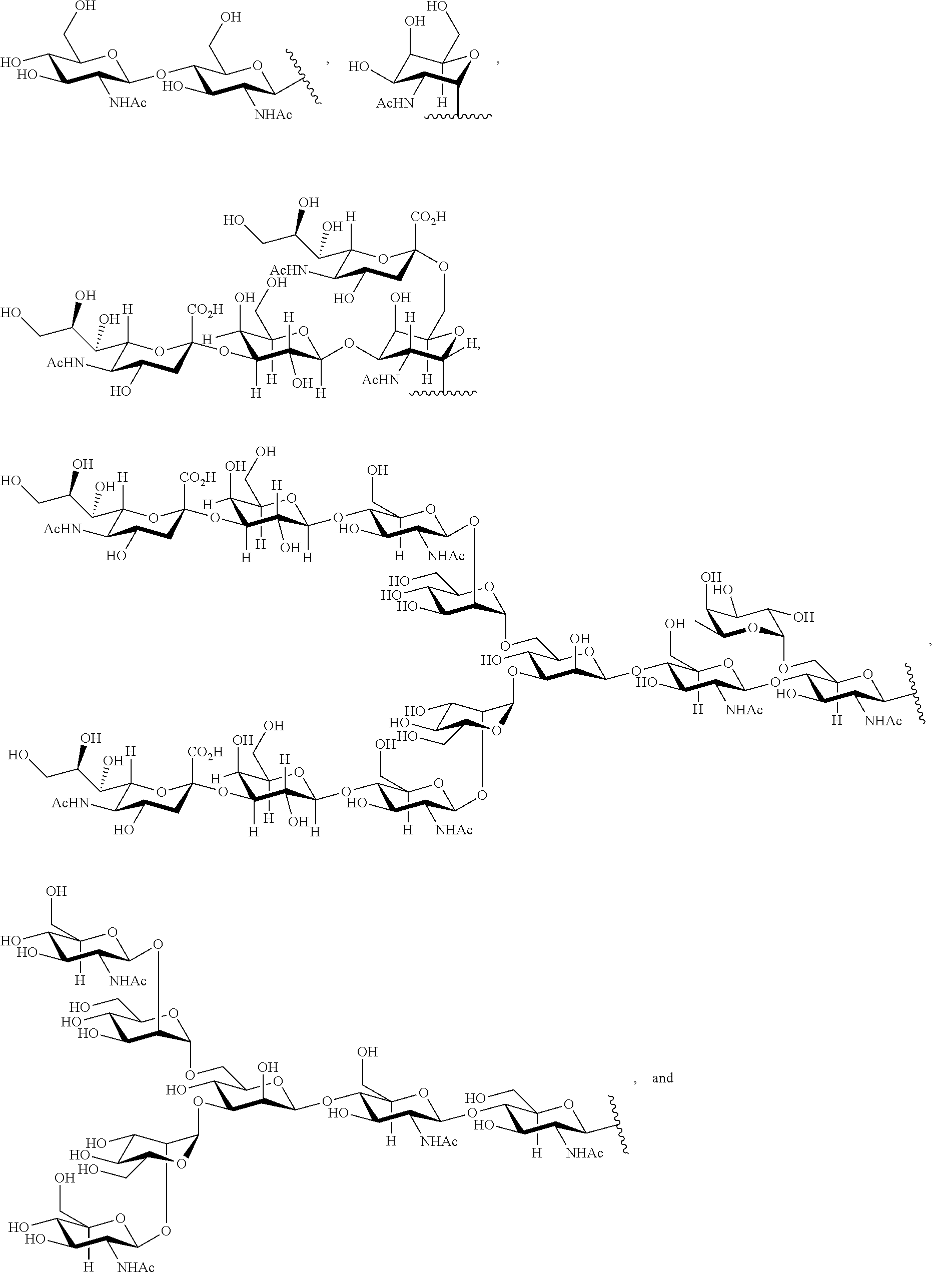

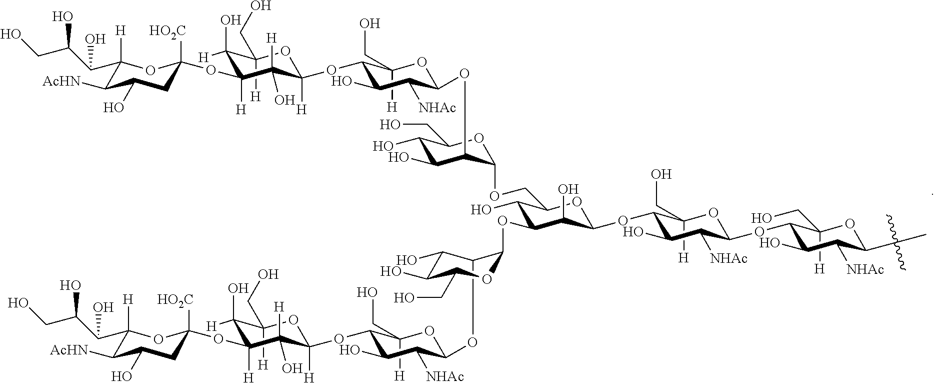

In some embodiments, each of Asn²⁴, Asn³⁸ and Asn⁸³ of the homogeneous, fully glycosylated erythropoietin has a glycan independently selected from:

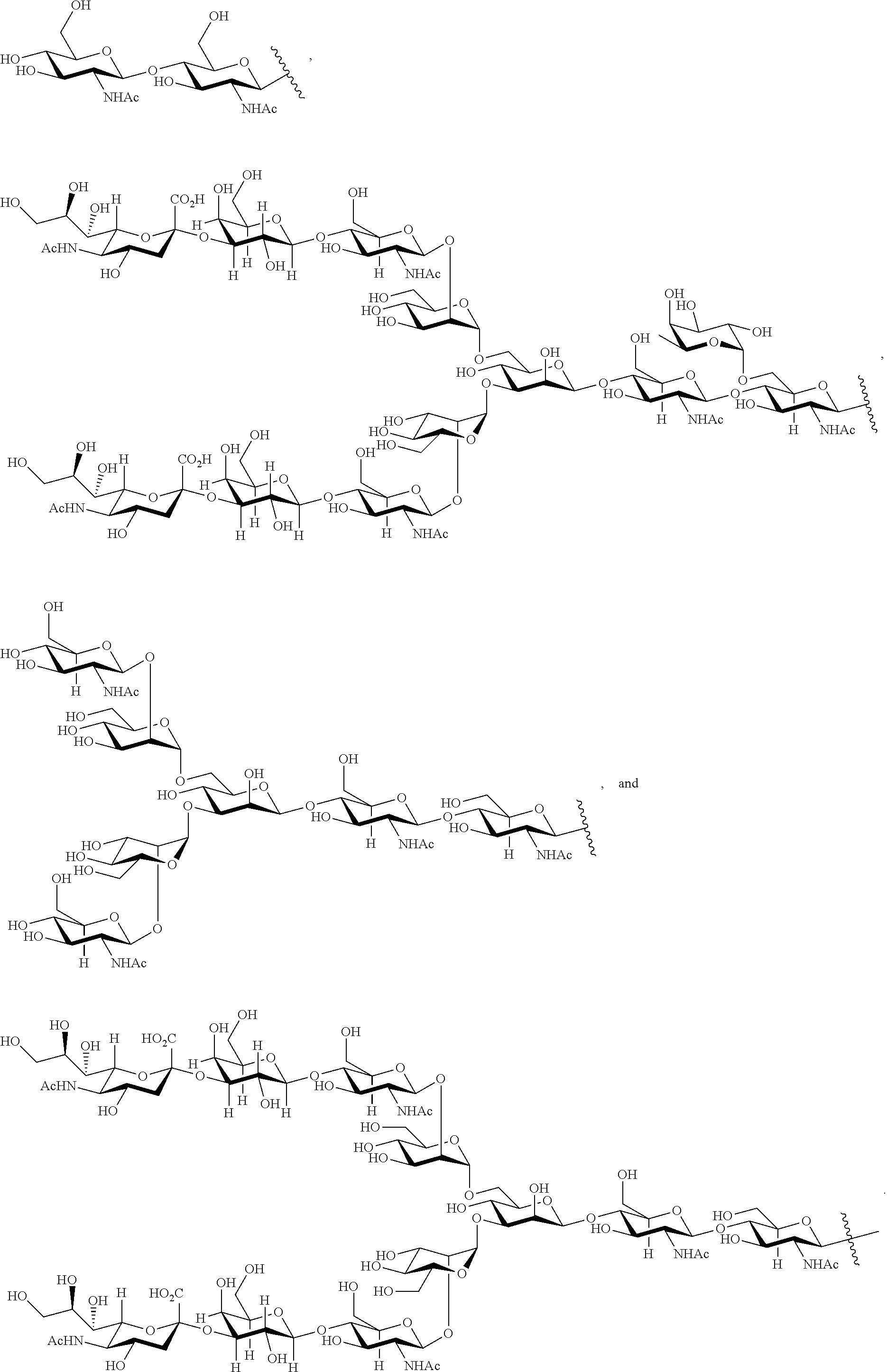

In some embodiments, Asn²⁴ of the homogeneous, fully glycosylated erythropoietin has a glycan selected from:

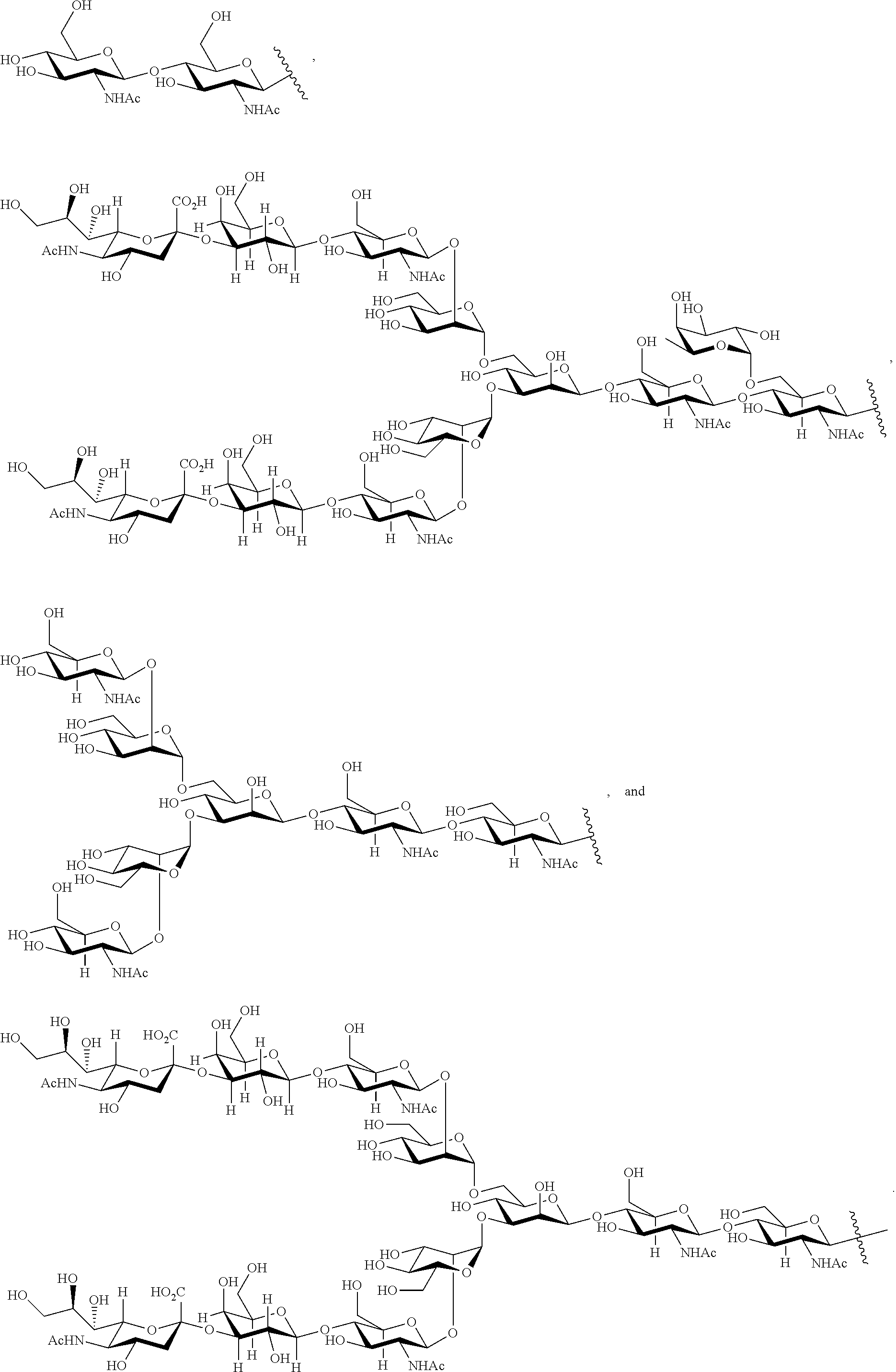

In some embodiments, Asn³⁸ of the homogeneous, fully glycosylated erythropoietin has a glycan selected from:

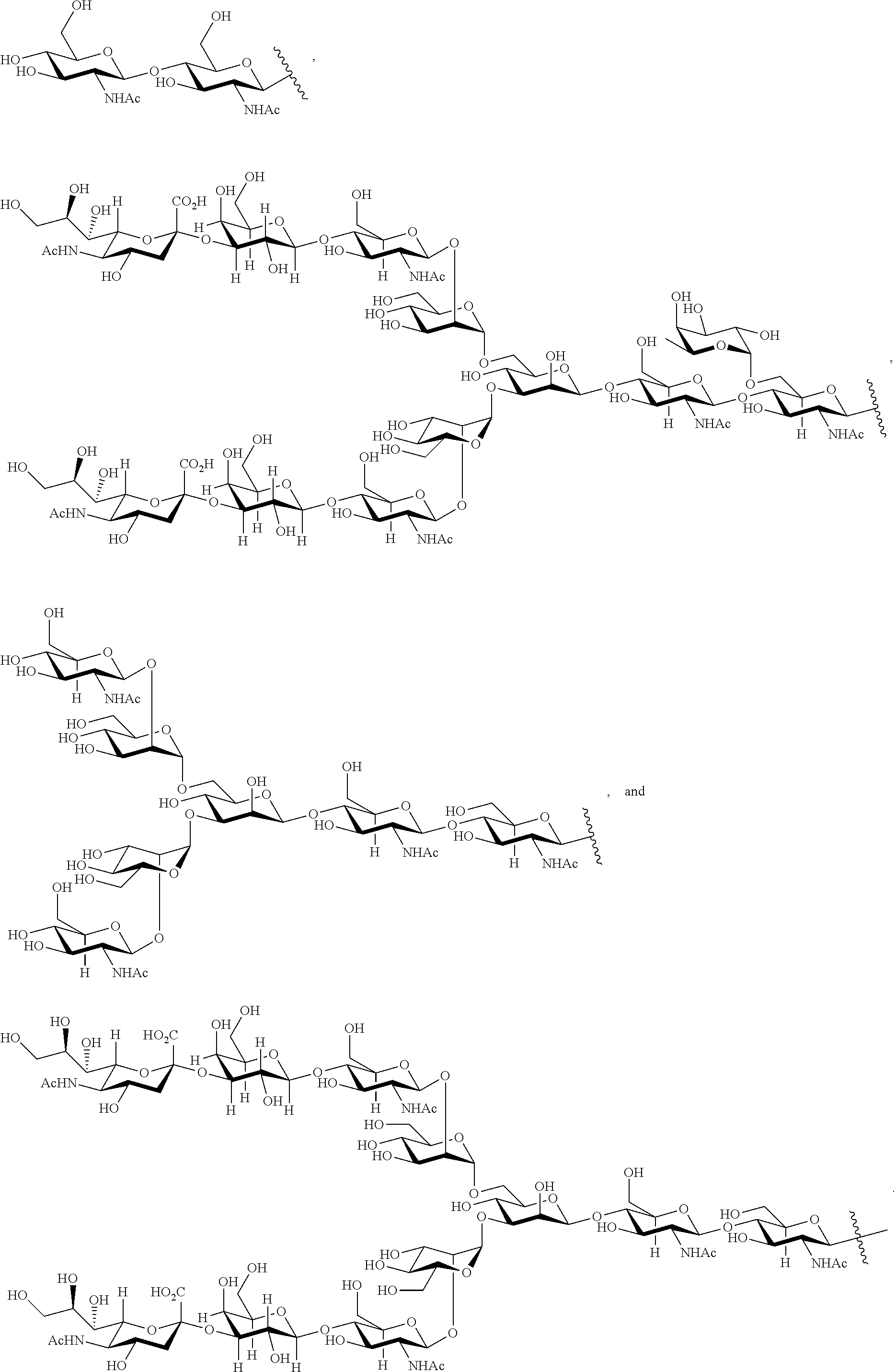

In some embodiments, Asn⁸³ of the homogeneous, fully glycosylated erythropoietin has a glycan selected from:

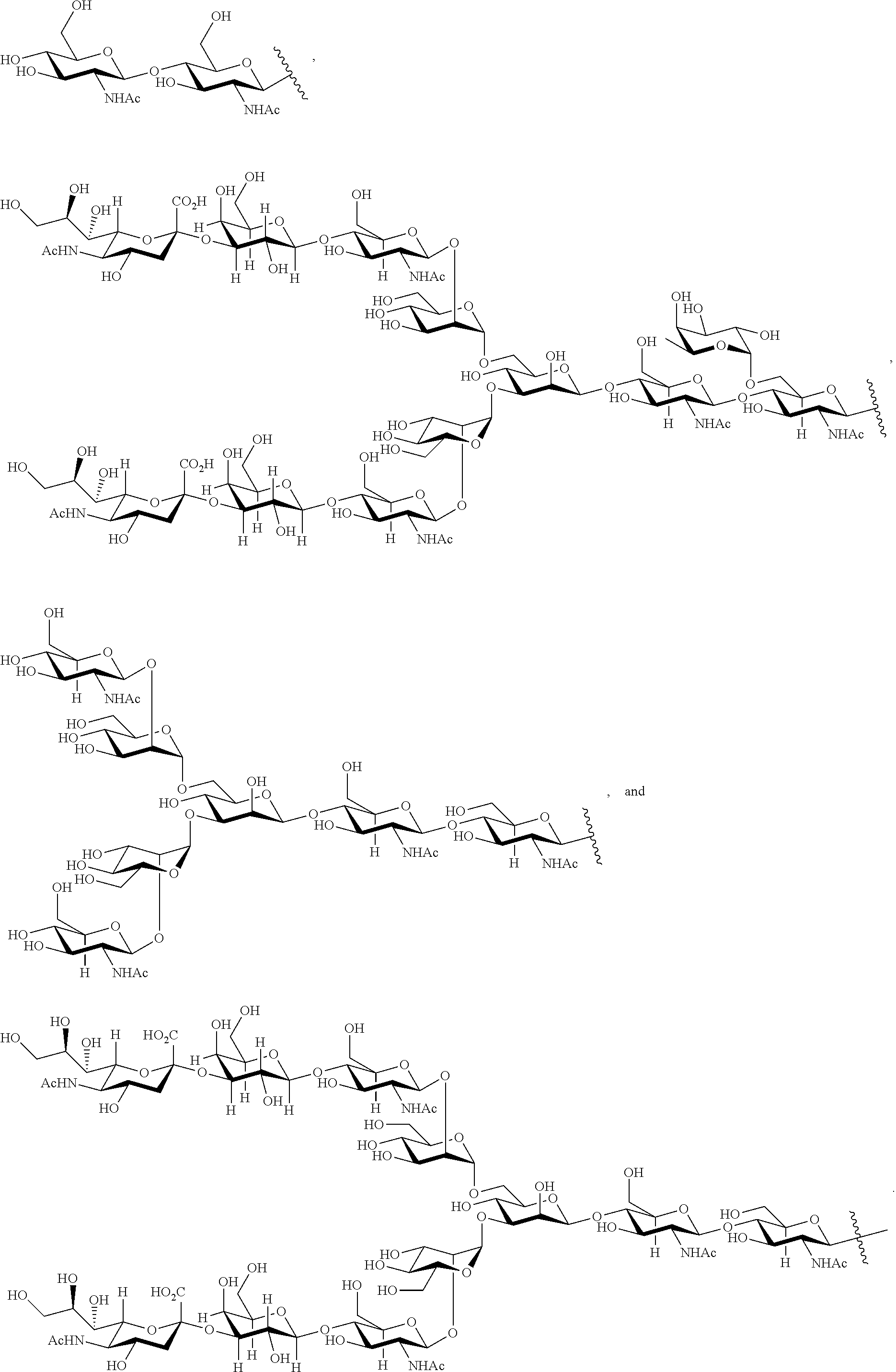

In some embodiments, Ser¹²⁶ of the homogeneous, fully glycosylated erythropoietin has a glycan selected from:

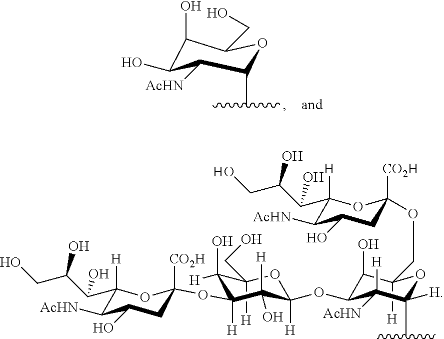

In some embodiments, each of Asn²⁴, Asn³⁸ and Asn⁸³ of the homogenous, fully glycosylated erythropoietin has a glycan independently selected from:

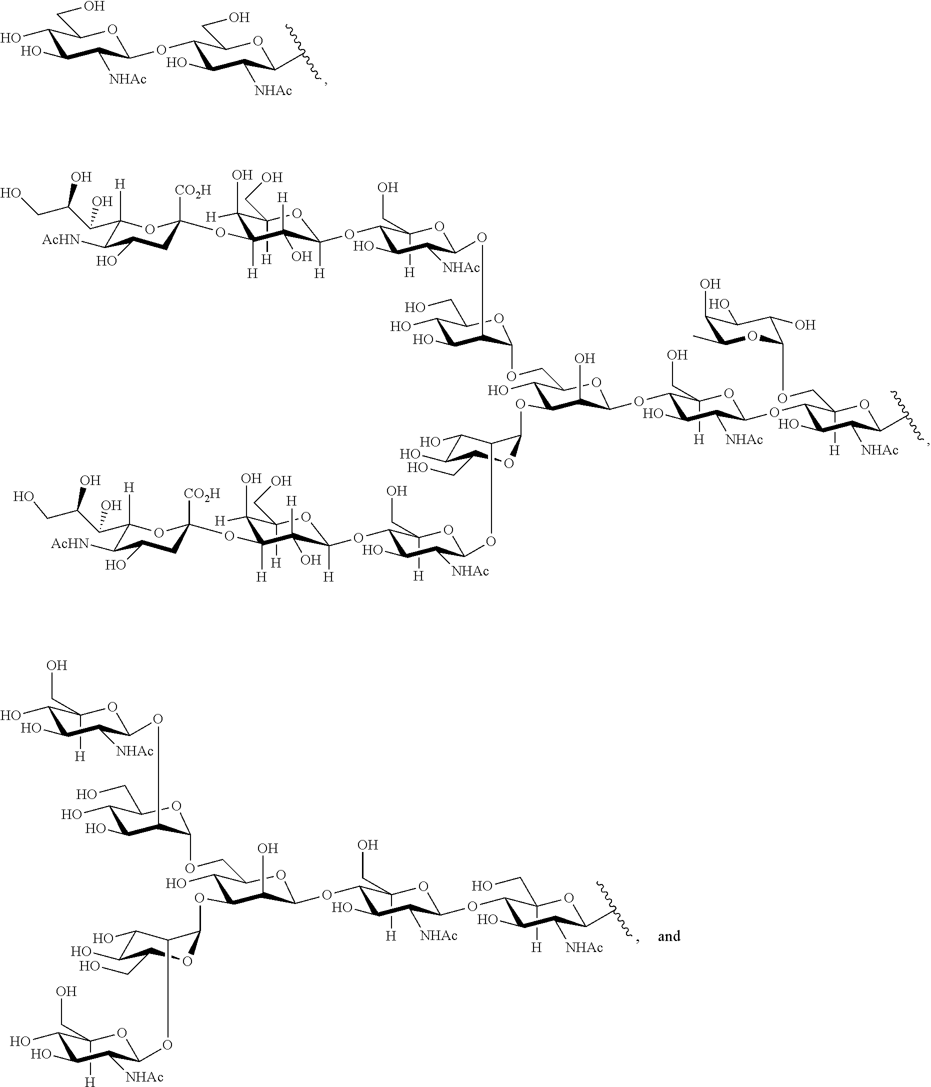

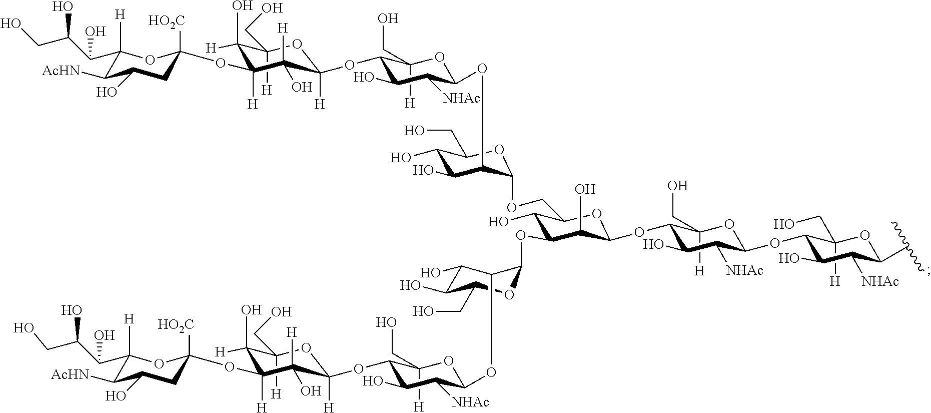

and Ser¹²⁶ of the homogeneous, fully glycosylated erythropoietin has a glycan selected from:

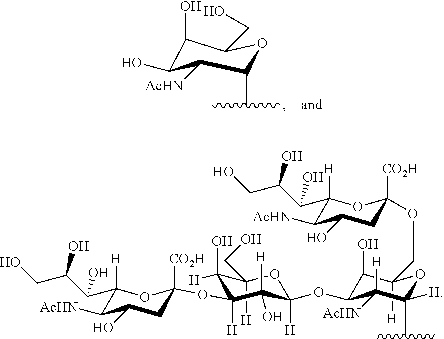

In some embodiments, Asn²⁴, Asn³⁸ and Asn⁸³ of the homogeneous, fully glycosylated erythropoietin have the same glycan.

In some embodiments, Asn²⁴, Asn³⁸ and Asn⁸³ of the homogenous, fully glycosylated erythropoietin have a glycan selected from:

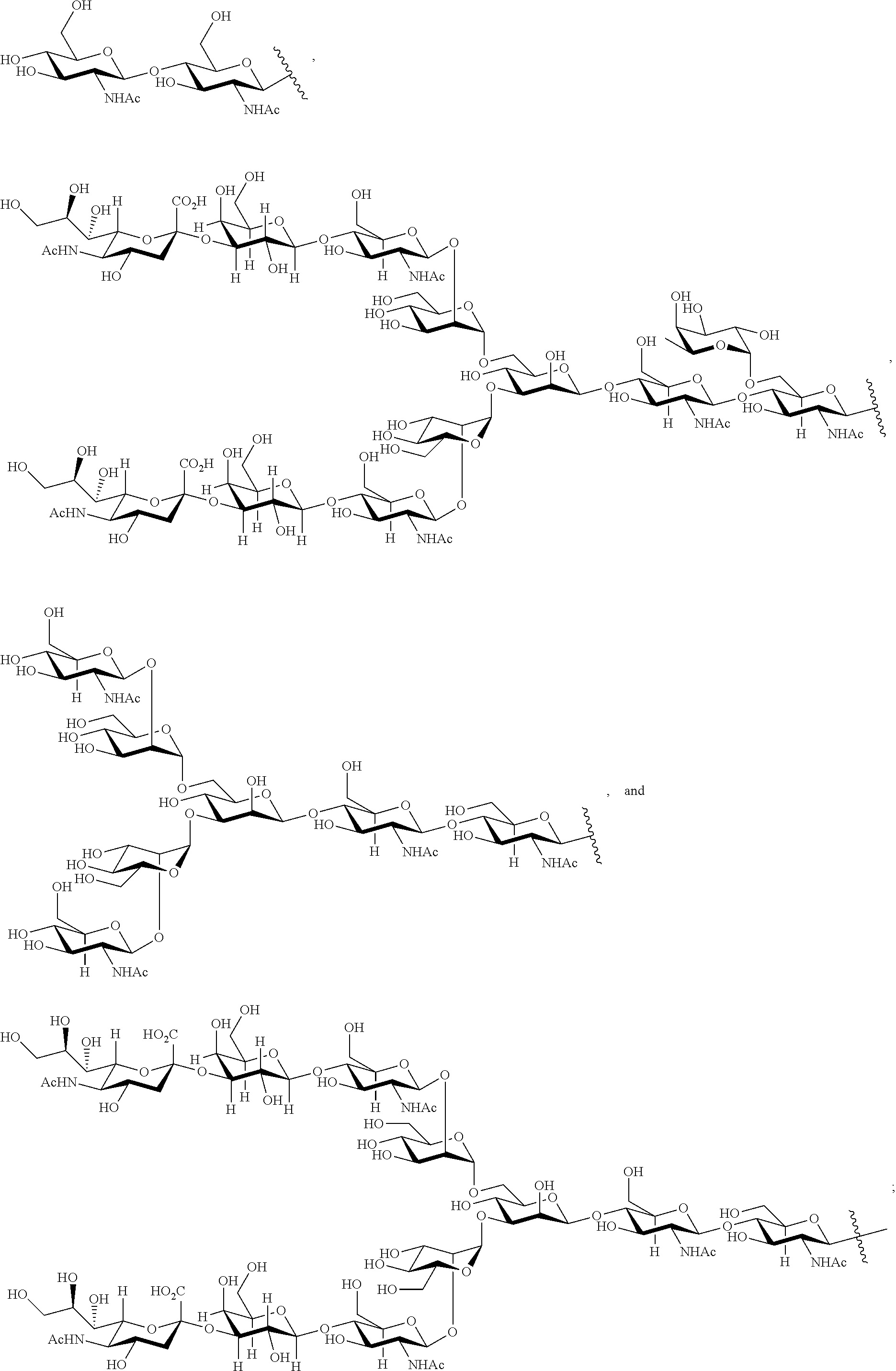

and Ser¹²⁶ of the homogeneous, fully glycosylated erythropoietin has a glycan selected from:

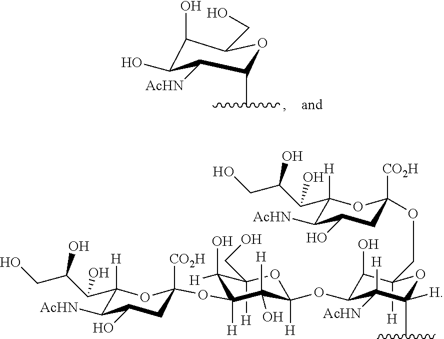

Exemplary homogeneous, fully glycosylated erythropoietins are depicted below:

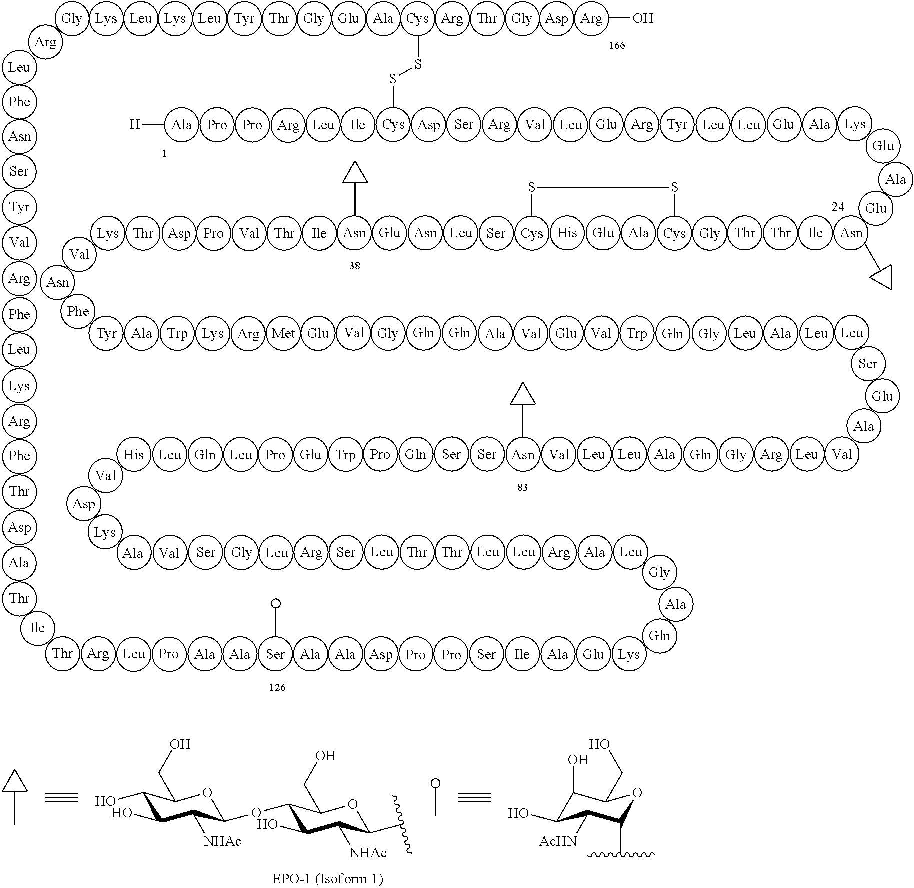

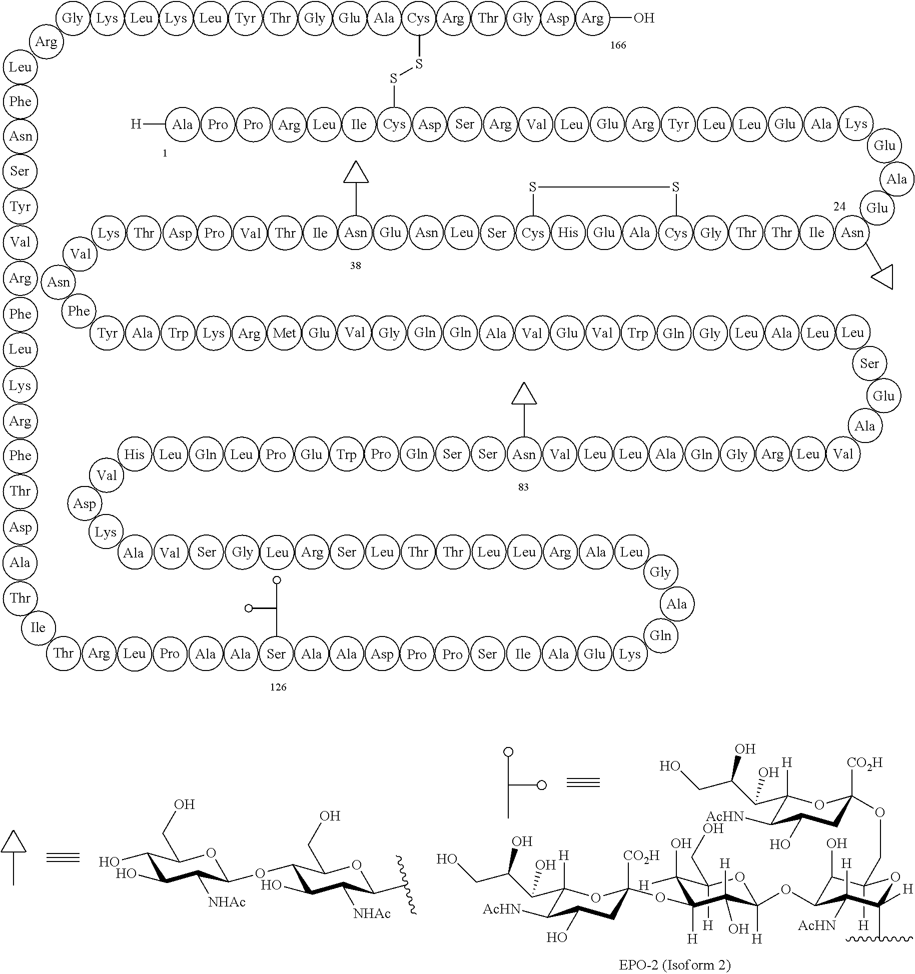

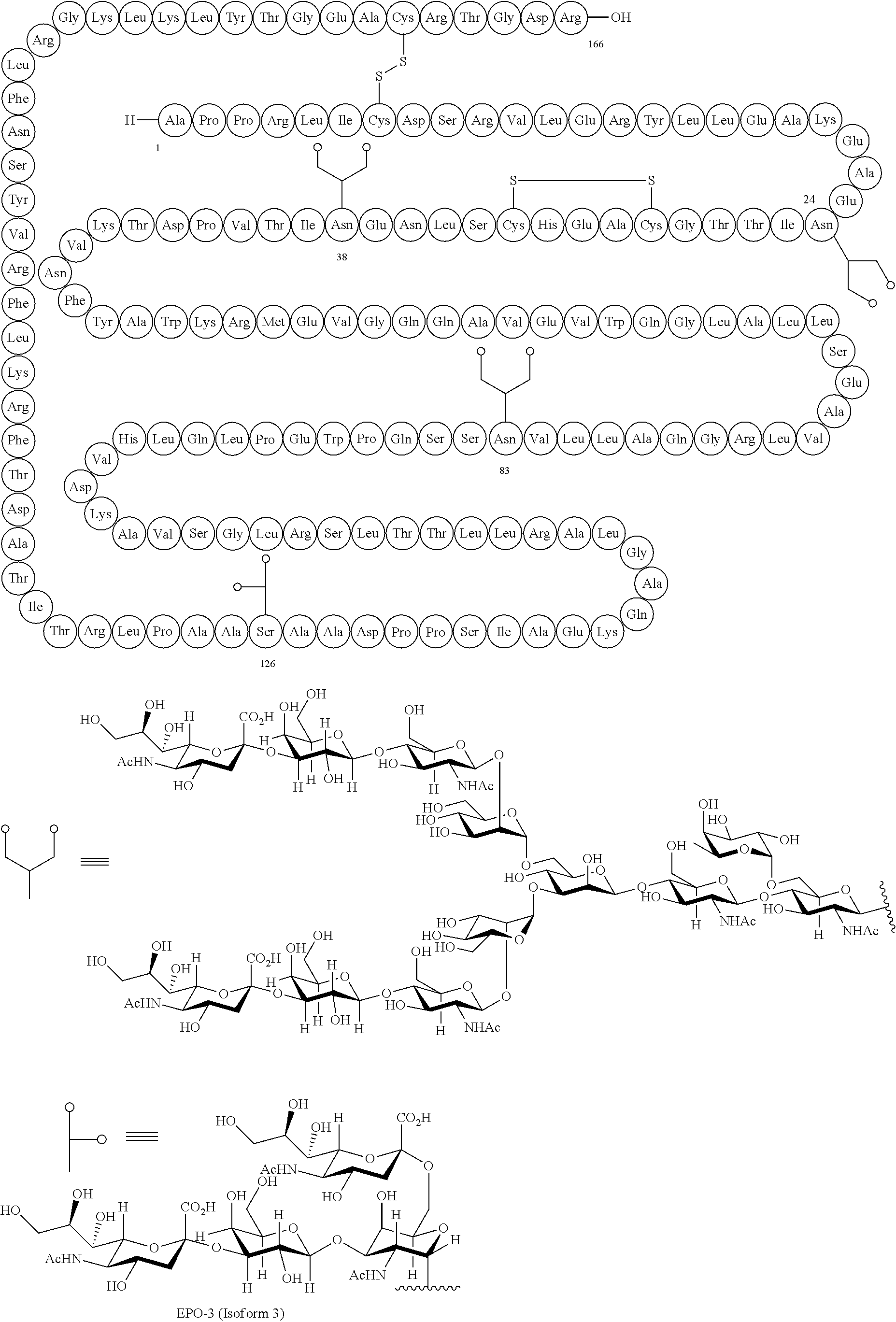

In some embodiments, the homogeneous, fully-glycosylated erythropoietin has mutations in its primary amino acid sequence. In some embodiments, the homogeneous, fully-glycosylated erythropoietin has mutations in its primary amino acid sequence wherein Asn²⁴, Asn³⁸, Asn⁸³ and Ser¹²⁶ are not mutated. In some embodiments, the homogeneous, fully-glycosylated erythropoietin has 1-20 amino acid substitutions, additions, and/or deletions. In some embodiments, the homogeneous, fully-glycosylated erythropoietin has 1-20 amino acid substitutions, additions, and/or deletions wherein Asn²⁴, Asn³⁸, Asn⁸³ and Ser¹²⁶ are not mutated. In some embodiments, the homogeneous, fully-glycosylated erythropoietin has 1-15 amino acid substitutions, additions, and/or deletions. In some embodiments, the homogeneous, fully-glycosylated erythropoietin has 1-15 amino acid substitutions, additions, and/or deletions wherein Asn²⁴, Asn³⁸, Asn⁸³ and Ser¹²⁶ are not mutated. In some embodiments, the homogeneous, fully-glycosylated erythropoietin has 1-10 amino acid substitutions, additions, and/or deletions. In some embodiments, the homogeneous, fully-glycosylated erythropoietin has 1-10 amino acid substitutions, additions, and/or deletions wherein Asn²⁴, Asn³⁸, Asn⁸³ and Ser¹²⁶ are not mutated. In some embodiments, the homogeneous, fully-glycosylated erythropoietin has 1-5 amino acid substitutions, additions, and/or deletions. In some embodiments, the homogeneous, fully-glycosylated erythropoietin has 1-5 amino acid substitutions, additions, and/or deletions wherein Asn²⁴, Asn³⁸, Asn⁸³ and Ser¹²⁶ are not mutated. In some embodiments, provided erythropoietin mutants or variants are characterized in that they have at least 50%, at least 60%, at least 70%, at least 80%, at least 90%, at least 95%, at least 100%, or greater than 100% of the activity of homogenous or non-homogeneous (i.e., recombinant) fully-glycosylated erythropoietin.

In some embodiments, the present invention provides methods for preparing homogenously glycosylated erythropoietin. In some embodiments, the present invention provides methods for preparing homogenously, fully glycosylated full-length erythropoietin.

In some embodiments, the present invention provides methods for preparing homogenously, fully glycosylated full-length erythropoietin through chemical synthesis. In some embodiments, native chemical ligation and cysteine-free ligations based on a mild metal-free desulfurization protocol are employed in the chemical synthesis of homogenously, fully glycosylated erythropoietin.

In some embodiments, the present invention provides linear synthetic routes for homogeneous, fully glycosylated erythropoietin. In some embodiments, the present invention provides convergent synthetic routes for homogeneous, fully glycosylated erythropoietin. One synthetic route is depicted in Scheme 1, below, wherein

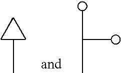

represent different glycans:

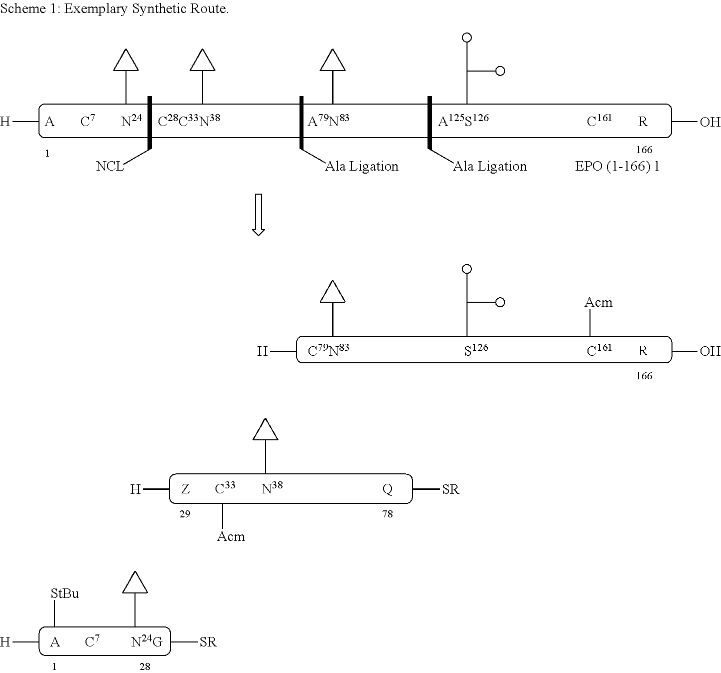

In some embodiments, the present invention further provides fragments that are useful in the synthetic route for homogeneous, fully glycosylated erythropoietin. In some embodiments, one or more of such fragments independently have mutations. In some embodiments, one or more of such fragments independently have 1-20 amino acid substitutions, additions, and/or deletions. In some embodiments, one or more of such fragments independently have 1-15 amino acid substitutions, additions, and/or deletions. In some embodiments, one or more of such fragments independently have 1-10 amino acid substitutions, additions, and/or deletions. In some embodiments, one or more of such fragments independently have 1-5 amino acid substitutions, additions, and/or deletions. In some embodiments, such fragments are useful for making homogenously glycosylated erythropoietin with mutations as described in this application.

Exemplary fragments useful for the synthesis of homogeneous, fully glycosylated erythropoietin are depicted below:

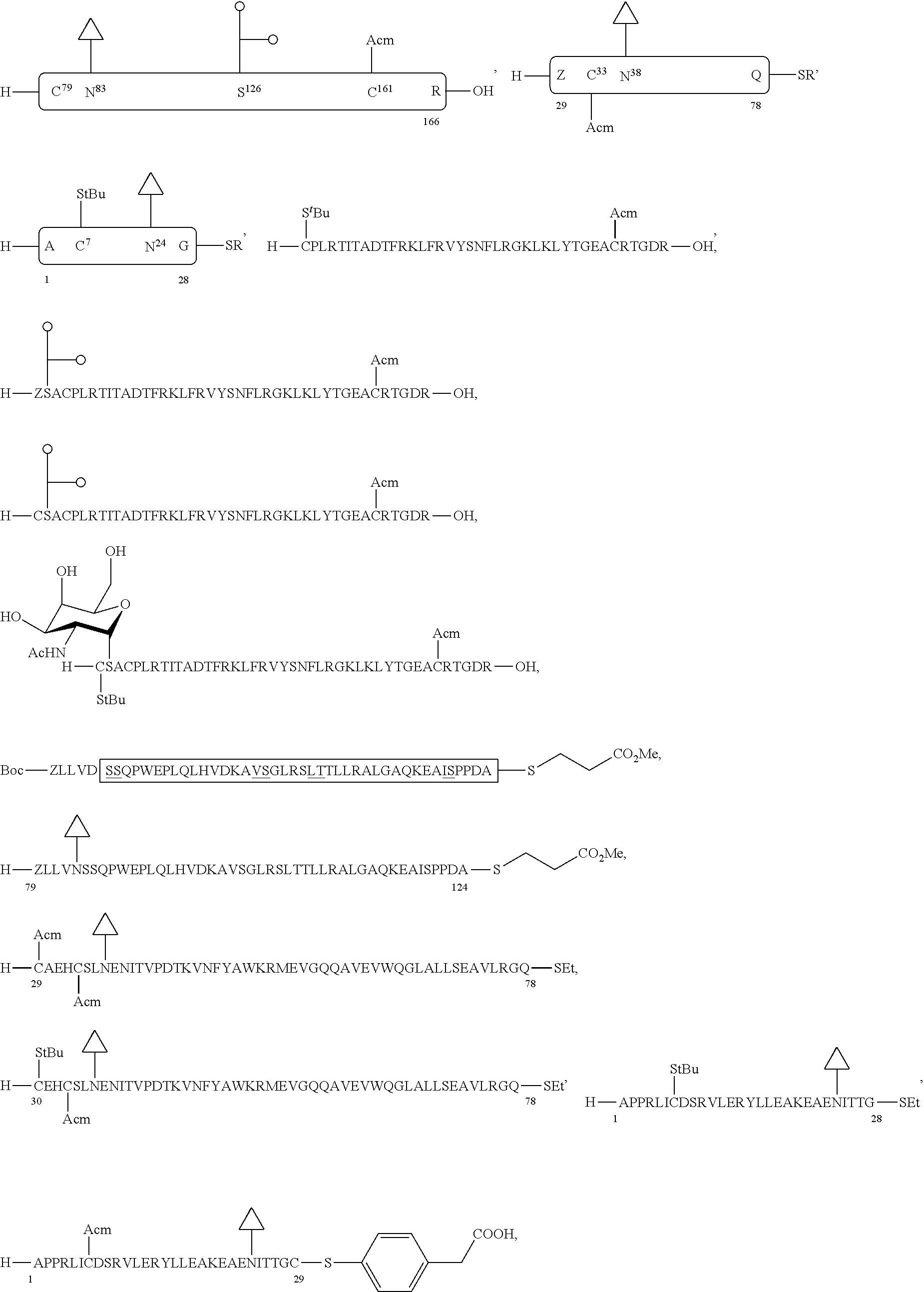

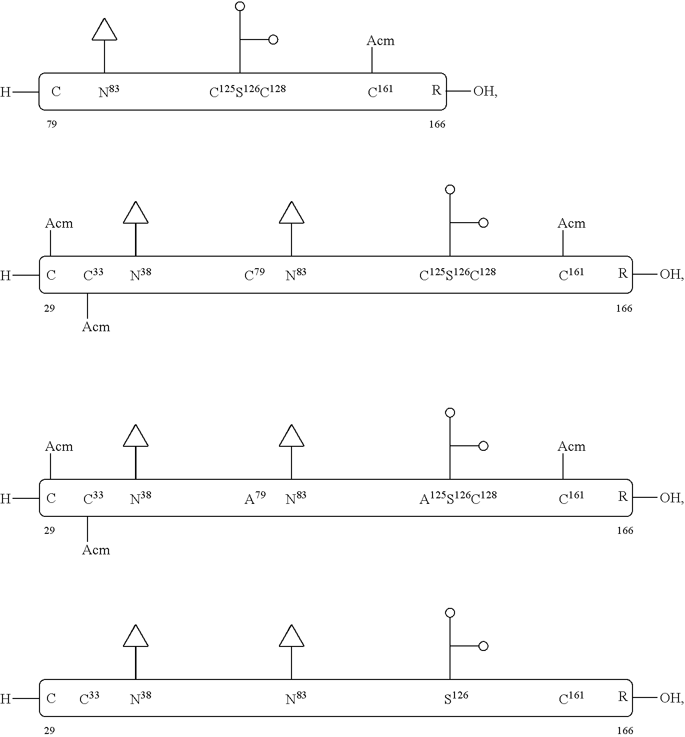

wherein

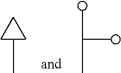

represent different glycans, “Acm” is acetomidomethyl, [symbol] side chain protected sequence, and [symbol] pseudoproline dipeptide.

In some embodiments, the present invention provides a method of preparing homogeneously glycosylated erythropoietin, the method comprising steps of ligating the glycosylated fragments EPO (1-28), EPO (29-78), EPO (79-124), EPO (125-166). In some embodiments, the fragments are ligated in a linear route. In some embodiments, the fragments are ligated in a linear route, wherein EPO (125-166) is first ligated with EPO (79-124), followed by EPO (29-78), and finally with EPO (1-28).

In some embodiments, the present invention provides a method of preparing homogeneously glycosylated erythropoietin, the method comprising steps of ligating the glycosylated fragments EPO (1-29), EPO (30-78), EPO (79-124), EPO (125-166). In some embodiments, the fragments are ligated in a convergent route. In some embodiments, the fragments are ligated in a convergent route, wherein EPO (1-29) is first ligated with EPO (30-78) to form EPO (1-78), followed by ligation with EPO (79-166) which is formed by ligation of EPO (79-124) and EPO (125-166).

In some embodiments, the present invention recognizes that certain amino acid residue(s) may hamper chemical synthesis of one or more fragments and/or fully-glycosylated erythropoietin. In certain embodiments, the present invention recognizes that certain amino acid residue(s) may hamper chemical synthesis of one or more fragments and/or fully-glycosylated erythropoietin due to aggregation. In certain embodiments, the present invention recognizes that certain amino acid residue(s) may hamper chemical synthesis of one or more fragments and/or fully-glycosylated erythropoietin due to the formation of secondary structures. In some embodiments, the present invention provides a solution to overcome such problems by the application of pseudoproline dipeptide. In some embodiments, pseudoproline dipeptides are used at S⁸⁴S⁸⁵, V⁹⁹S¹⁰⁰, L¹⁰⁵T¹⁰⁶ and I¹¹⁹S¹²⁰.

In some embodiments, native chemical ligation and cysteine-free ligations based on a mild metal-free desulfurization protocol are employed in the chemical synthesis of homogenously, fully glycosylated erythropoietin.

In some embodiments, the present invention recognizes that special solvents are required for certain steps of reactions. In some embodiments, the present invention recognizes that special solvents are required for certain reagents and/or products. In some embodiments, the present invention recognizes that special solvents are required for certain reagents and/or products due to low solubility. In some embodiments, trifluoroethanol is used as a solvent for reagents with poor solubility. In some embodiments, trifluoroethanol is used for

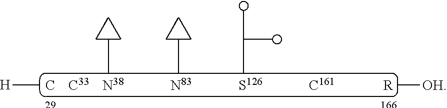

In some embodiments, the present invention provides methods to study the structure-function relationships of homogeneously glycosylated erythropoietin. In some embodiments, the present invention provides methods to study the structure-function relationships of erythropoietin glycoforms using homogenously glycosylated erythropoietin. In some embodiments, the present invention provides methods to study the structure-function relationships of erythropoietin glycoforms using homogenous, fully glycosylated full-length erythropoietin.

### EXEMPLIFICATION

The representative examples which follow are intended to help illustrate the invention, and are not intended to, nor should they be construed to, limit the scope of the invention. It will be appreciated by one of ordinary skill in the art that the present invention encompasses the use of various alternate protecting groups and glycans known in the art to make many further embodiments in this application in addition to those shown and described herein. Those protecting groups and glycans used in the disclosure including the Examples below are illustrative.

Methods for preparing glycopeptides (e.g., O- or N-linked glycopeptides) and for conjugating peptides and glycopeptides to carriers are known in the art. For example, guidance may be found in U.S. Pat. No. 6,660,714; U.S. patent application Ser. Nos. 09/641,742, 10/209,618, 10/728,041 and 12/296,608; U.S. Provisional Patent Application Nos. 60/500,161, 60/500,708, 60/560,147, 60/791,614 and 60/841,678; and International Patent Application Nos.: PCT/US03/38453, PCT/US03/38471, PCT/US2004/29047 and PCT/US07/08764; each of the above-referenced patent documents are hereby incorporated by reference herein.

### 1. Synthesis of EPO-2 (1)—Description

As shown in Scheme 2, unfolded EPO primary structure EPO-2 (1) could be dissected into four glycopeptide segments. A linear strategy using two alanine ligations and a final native chemical ligation (NCL) may assemble the full sequence from the C-terminus of the protein. In order to differentiate the “to be dethiylated” and the native cysteine residues, protection with acetomidomethyl (Acm) at Cys³³ and Cys¹⁶¹ groups was used.

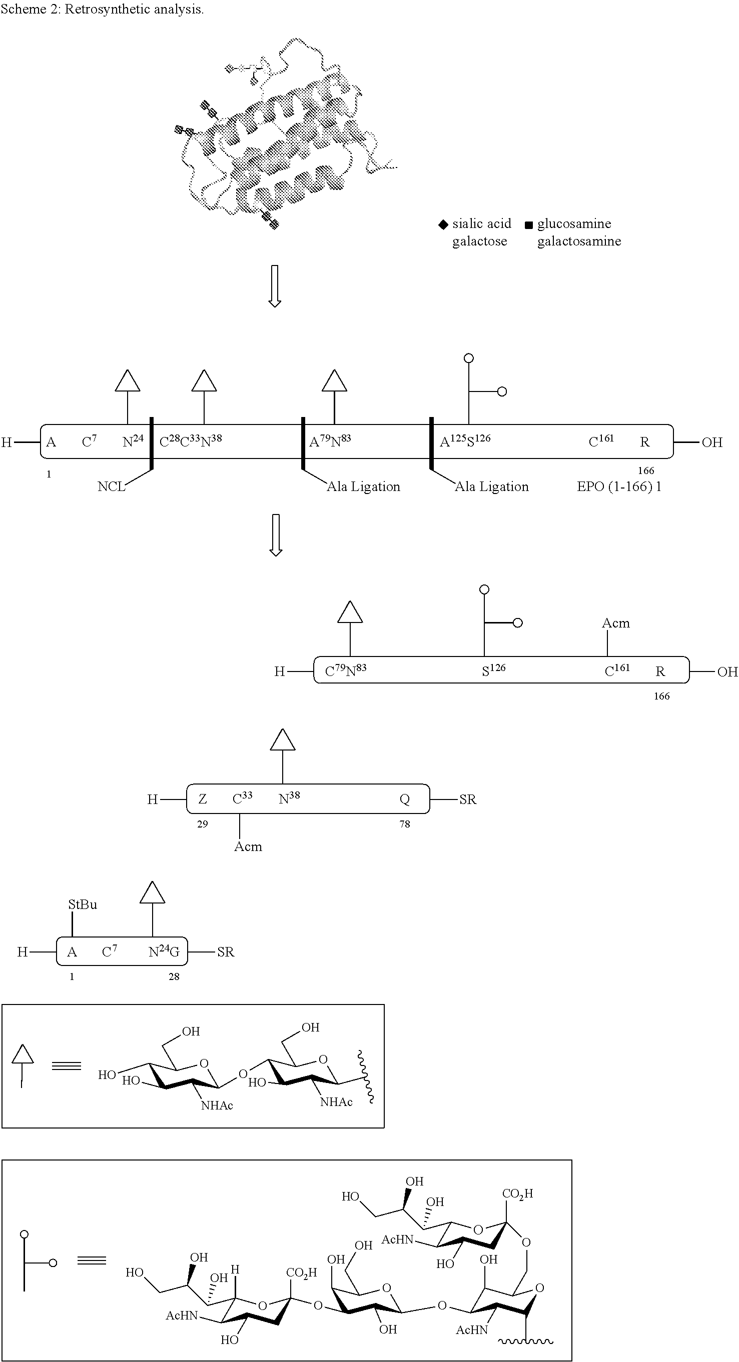

We first prepared EPO (125-166) containing the only O-linked glycan of the protein. Previously, we have demonstrated that complex O-linked Ser glycoside, such as glycophorin (D. B. Thomas, R. J. Winzler, J. Biol. Chem. 1969, 244, 5943-5946), could be utilized in efficient synthesis of α-O-linked glycopeptides from a fully protected cassette (J. B. Schwarz, S. D. Kuduk, X.-T. Chen, D. Sames, P. W. Glunz, S. J. Danishefsky, J. Am. Chem. Soc. 1999, 121, 2662-2673). Global deprotection using sodium hydroxide followed by the reaction with Fmoc-thiazolidine succinimide ester 3 under basic conditions afforded glycopeptide 4. By coupling with alanine (2-ethyldithiolphenyl)ester 5, compound 4 was elongated to tripeptide 6 bearing a more durable thioester equivalent (Scheme 3, Warren, J. D.; Miller, J. S.; Keding, S. J.; Danishefsky, S. J. J. Am. Chem. Soc. 2004, 126, 6576; Chen, G.; Warren, J. D.; Chen. J.; Wu, B.; Wan, Q.; Danishefsky, S. J. J. Am. Chem. Soc. 2006, 128, 7460).

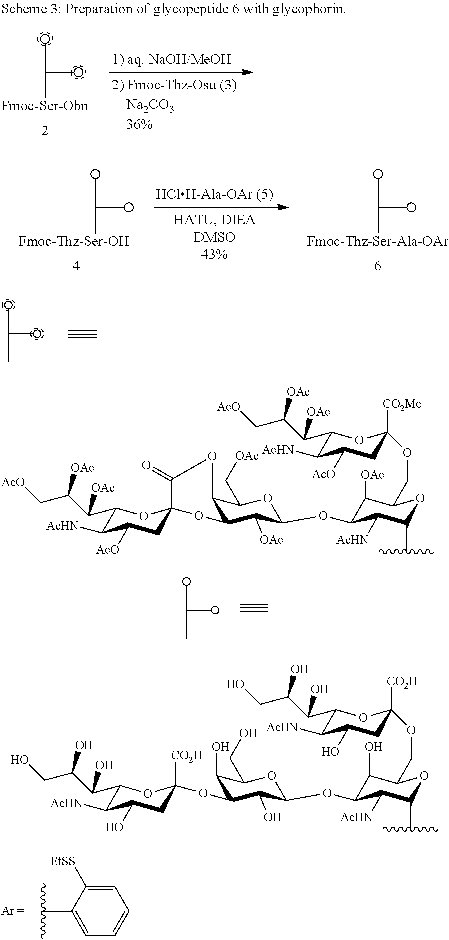

With glycopeptide 6 in hand, we next conducted the NCL reaction with peptide 7 (Scheme 4, A), which was prepared directly by solid-phase peptide synthesis (SPPS) using an Fmoc strategy. In the event, the ligation of 6 and 7 proceeded smoothly. After removal of the Fmoc group followed by thiazolidine ring opening, EPO (125-166) 9 with glycophorin was obtained in good yield. On the other hand, glycopeptide 10 with N-acetylgalactosamine could be prepared from serine cassette 11 via SPPS followed by deprotections (Scheme 4, B).

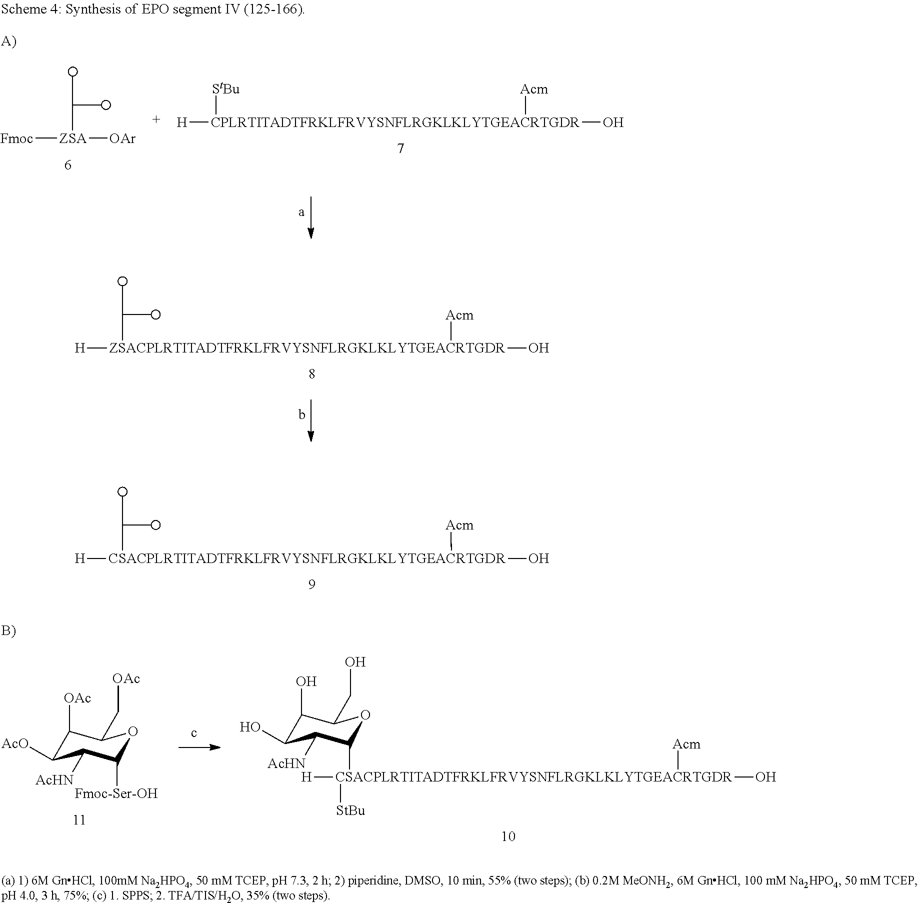

For glycopeptide segments with N-linked glycosylation site, a unified approach was utilized. From side chain protected peptide 12, HATU-mediated glycosylation with chitobiose, followed by global deprotection, afforded glycopeptide segment 13 EPO (Ala⁷⁹-Ala¹²⁴) in good isolated yield after RP-HPLC purification (Scheme 5). In a similar manner, EPO segments II (Scheme 6, 14, Cys²⁹-Gln78; or 15, Cys³⁰-Gln78), and I (16, Ala¹-Gly²⁸; or 17, Ala¹-Cys²⁹) were prepared accordingly.

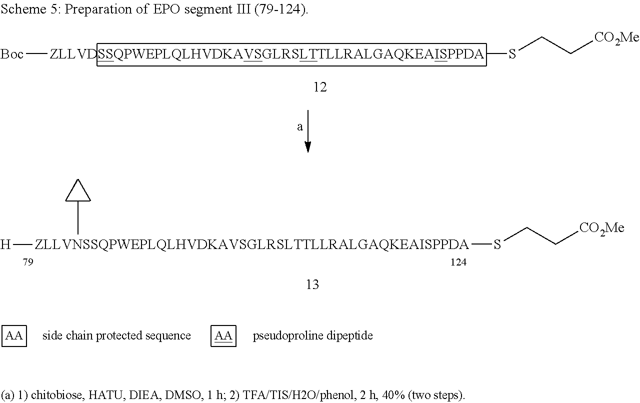

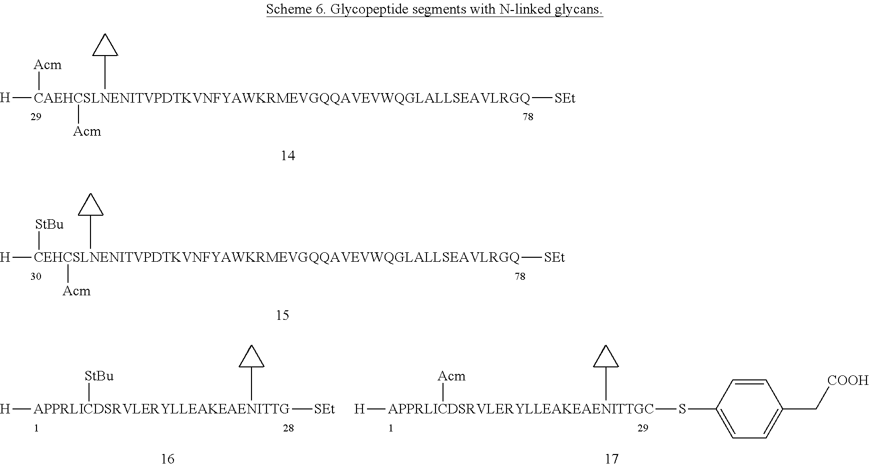

With all required glycopeptide segments in hand, we next conducted the ligation reactions for the assembly of EPO (1-166) (Scheme 7). Under standard NCL conditions, ligation of glycopeptides 9 and 13 cleanly afforded compound 18. In a similar manner, glycopeptide 14 was also incorporated to afford peptide 19, which contains three cysteine residues that need to be converted into native alanines in the desired peptide segment 20. Utilizing our previously developed metal-free desulfurization protocol (Wan, Q.; Danishefsky, S. J. Angew. Chem., Int. Ed. 2007, 46, 9248-9252), all three thiol groups were completely removed leading to 20 with all three required Ala residues in the native EPO sequence. After the removal of Acm groups according to the literature reported protocol (Liu, S.; Pentelute, B. L.; Kent, S. B. H. Angew. Chem., Int. Ed. 2012, 51, 993-999), the final ligation of EPO (29-166) 21 and EPO (1-28) 16 successfully produced the primary structure of erythropoietin 1 with all four required glycosylation. Noticeably, EPO (29-166) showed poor solubility especially peptide 21, thus the use of trifluoroethanol (TFE) as cosolvent in the final step was crucial for the reaction to proceed (Naider, F.; Estephan, R.; Englander, J.; Suresh babu, V. V.; Arevalo, E.; Samples, K.; Becker, J. M. Pept. Sci. 2004, 76, 119-128).

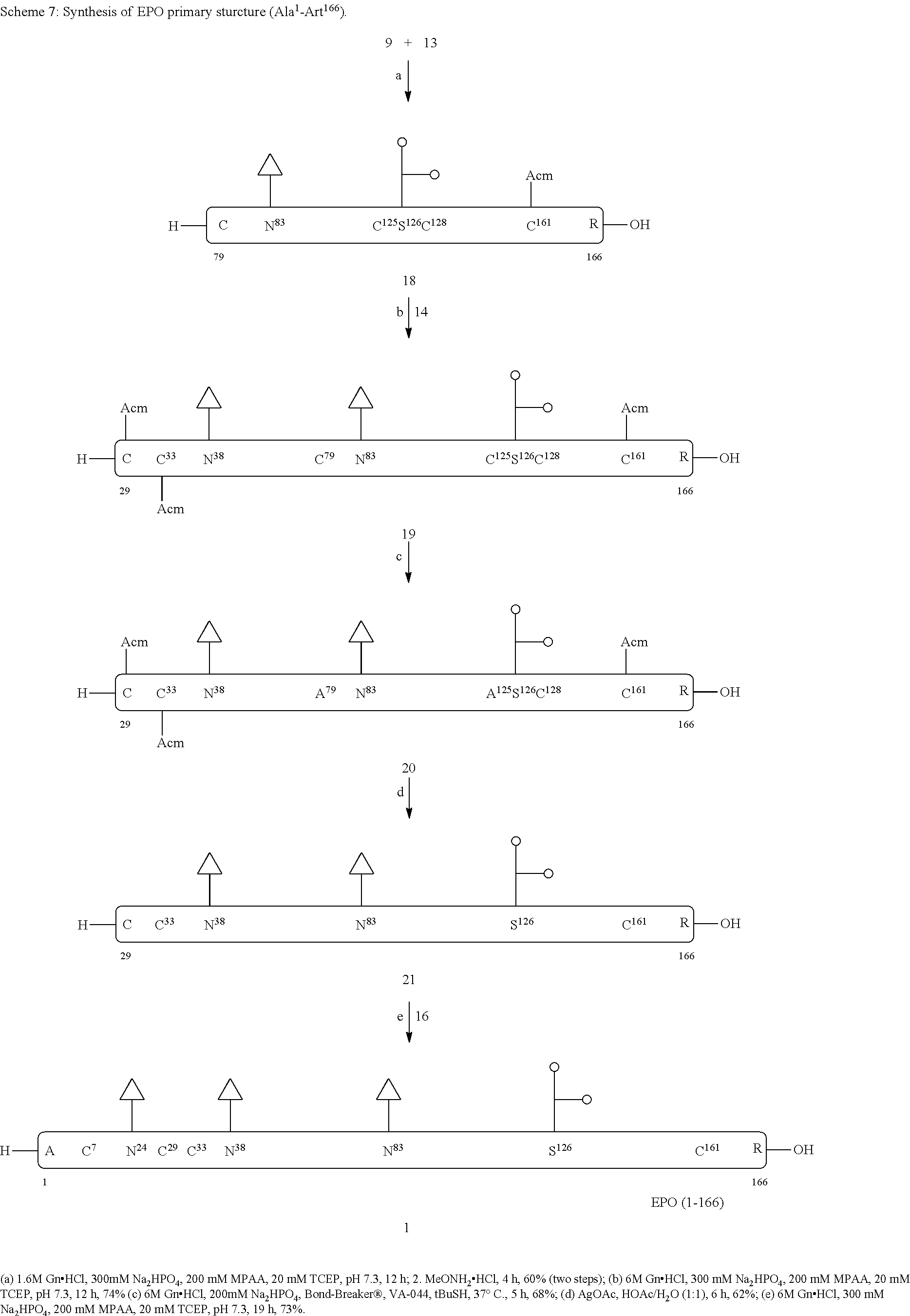

As glycopeptide sequence 1 was successfully prepared through a linear synthetic route, an alternative convergent route was also utilized (Scheme 8). In the event, kinetic native chemical ligation of slightly modified segments 15 and 17 ((a) Bang, D.; Pentelute, B. L.; Kent, S. B. H. Angew. Chem. Int. Ed. 2006, 45, 3985-3988; (b) Torbeev, V. Y.; Kent, S. B. H. Angew. Chem. Int. Ed. 2007, 46, 1667-1670; (c) Durek, T.; Torbeev, V. Y.; Kent, S. B. H. Proc. Natl. Acad. Sci. USA 2007, 104, 4846-4851), followed by in situ activation of Gln⁷⁸ alkylthioester using mercaptophenylacetic acid (MPAA) in the presence of glycopeptide 18 (Johnson, E. C. B.; Kent, S. B. H. J. Am. Chem. Soc. 2006, 128, 6640-6646), generated the protected EPO full sequence 22 in one-pot. After dialysis using a centrifugal unit, the crude mixture was directly subjected to standard desulfurization conditions, which afforded desired glycopeptide 23 in good yield. Final treatment of 23 with AgOAc in acetic acid solution removed all four Acm protecting groups leading to the generation of product 1.

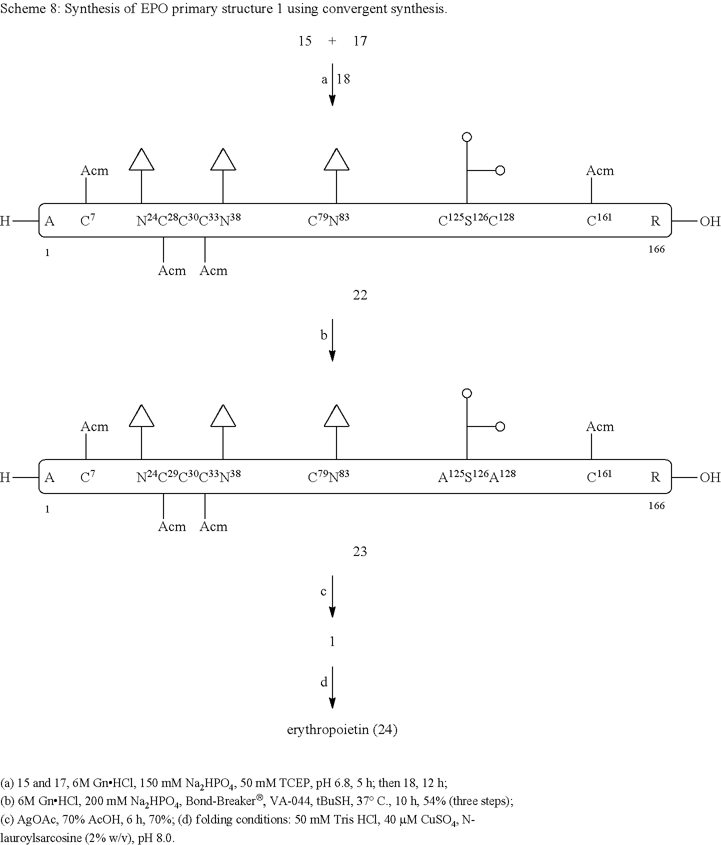

### 2. Folding and Activity of Synthetic, Homogeneously Glycosylated Erythropoietin

Folding experiment was conducted following literature reported protocol using CuSO₄ as oxidant. The obtained protein 24 was evaluated in a cell proliferation assay. The TF-1 cell line established from a patient with erythroleukemia undergoes short term proliferation and terminal erythroid differentiation in response to erythropoietin (Kitamura, T.; Tange, T.; Terasawa, T.; Chiba, S.; Kuwaki, T.; Miyagawa, K.; Piao, Y F.; Miyazono, K.; Urabe, A.; Takaku, F. J. Cell Physiol. 1989, 140, 323-34). The activity of synthetic EPO (22) was compared to COS cell-derived clinical grade EPO (Procrit®) over a dose range of 0.01-30.00 ng/ml using 5000 TF-1 cells/60 μl of IMDM medium containing 20% Serum Replacement in 384-wells plate in triplicates. After 3 days incubation, the cultures were pulsed with Alarma Blue overnight and fluorescence intensity measured using a Synergy H1 platereader (BioTek).

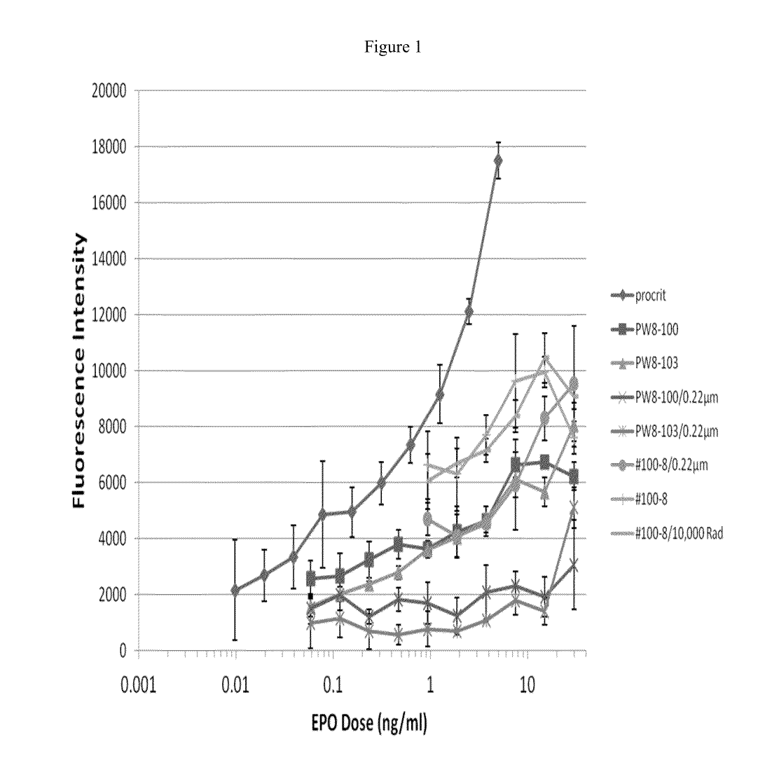

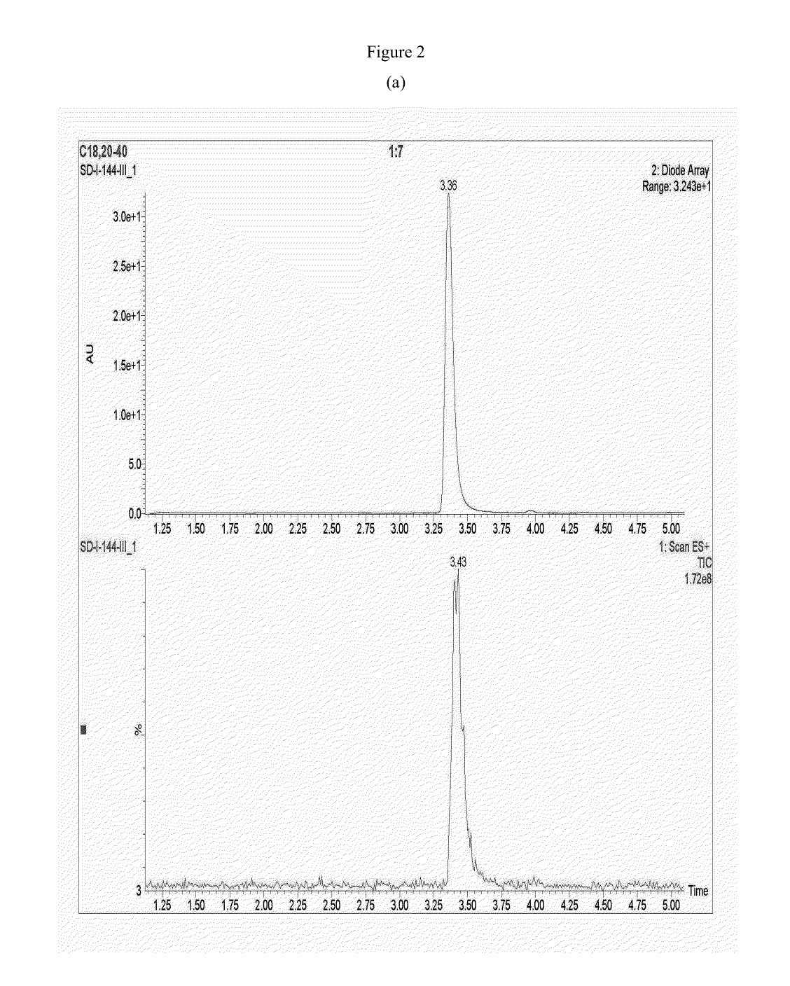

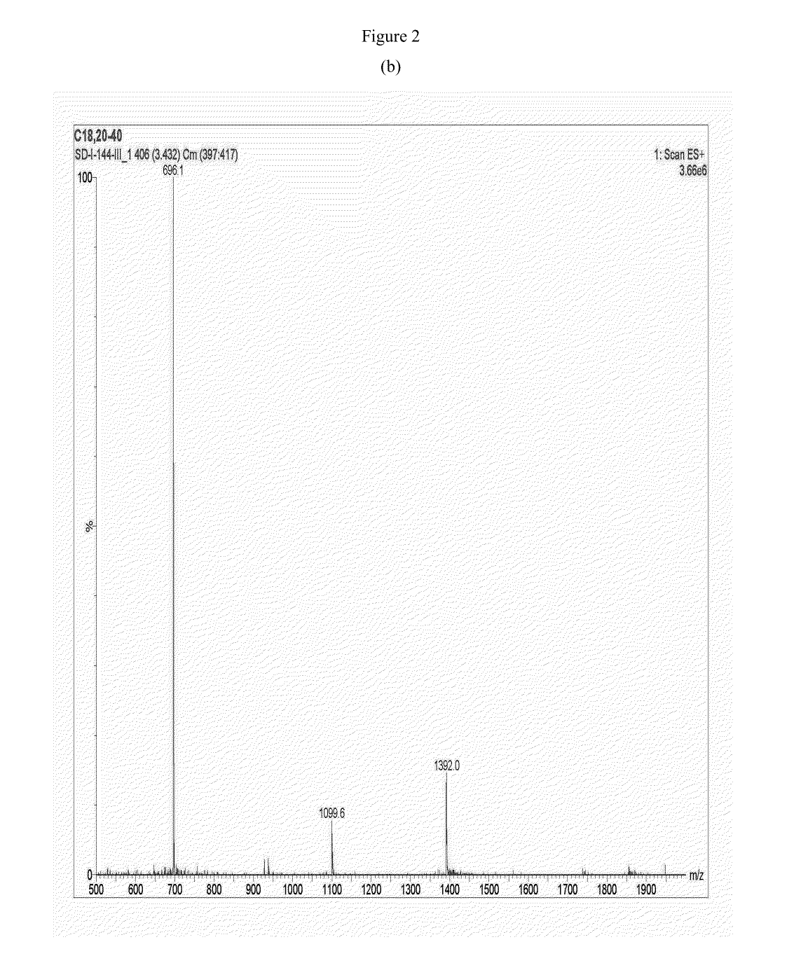

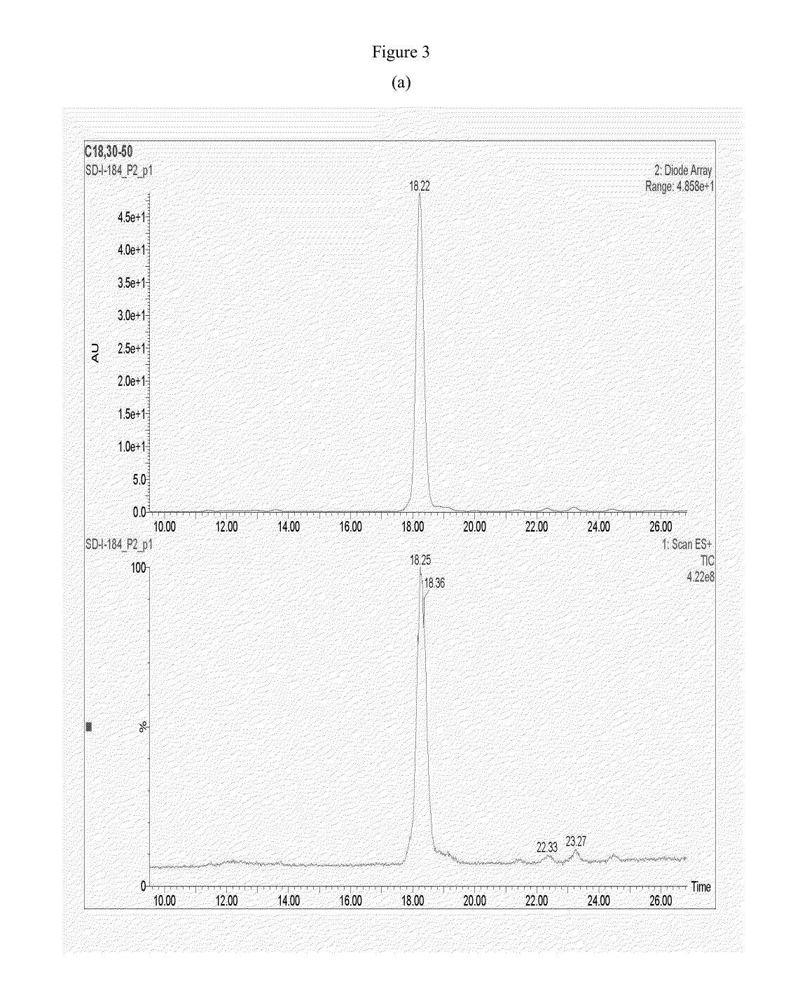

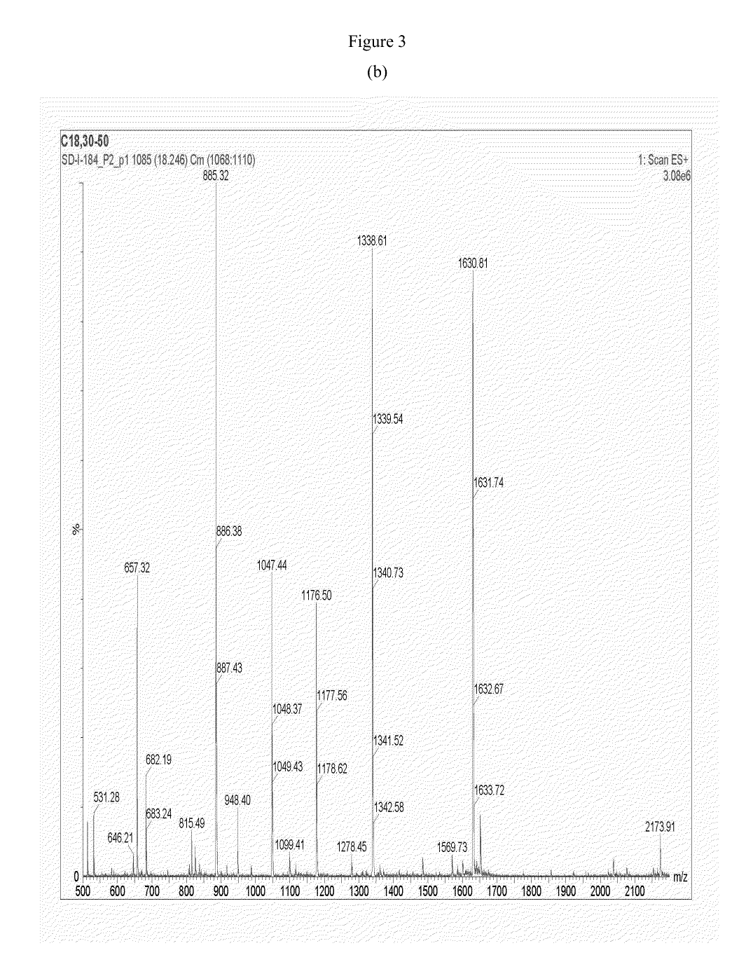

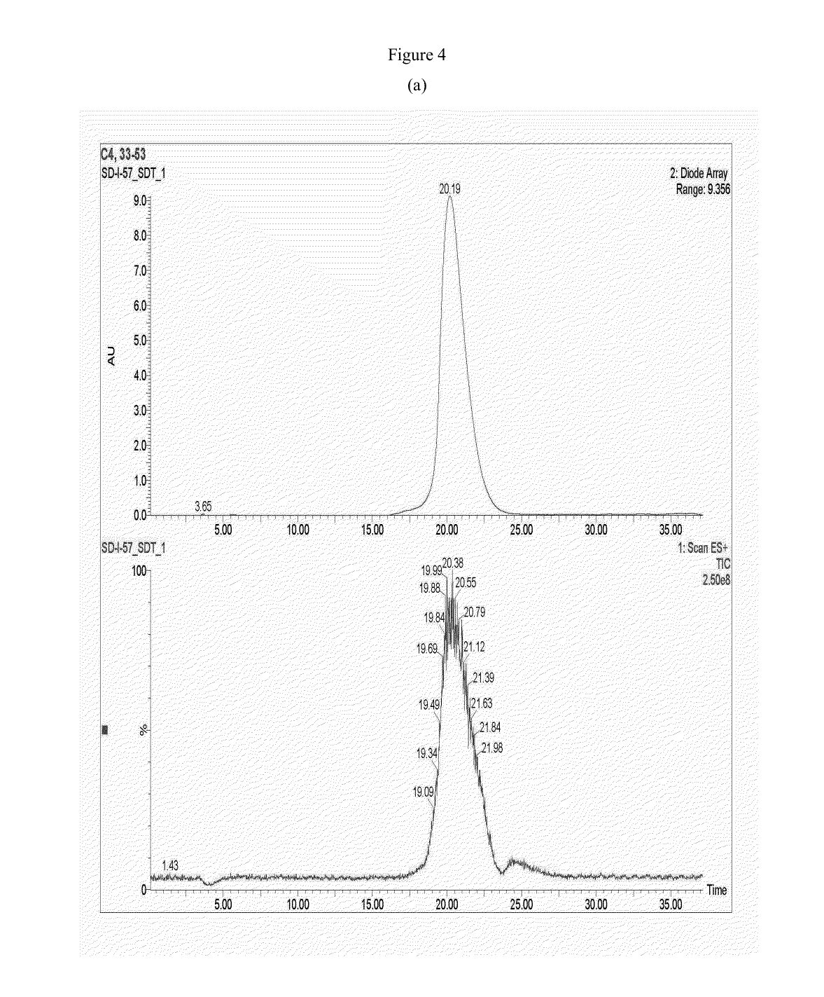

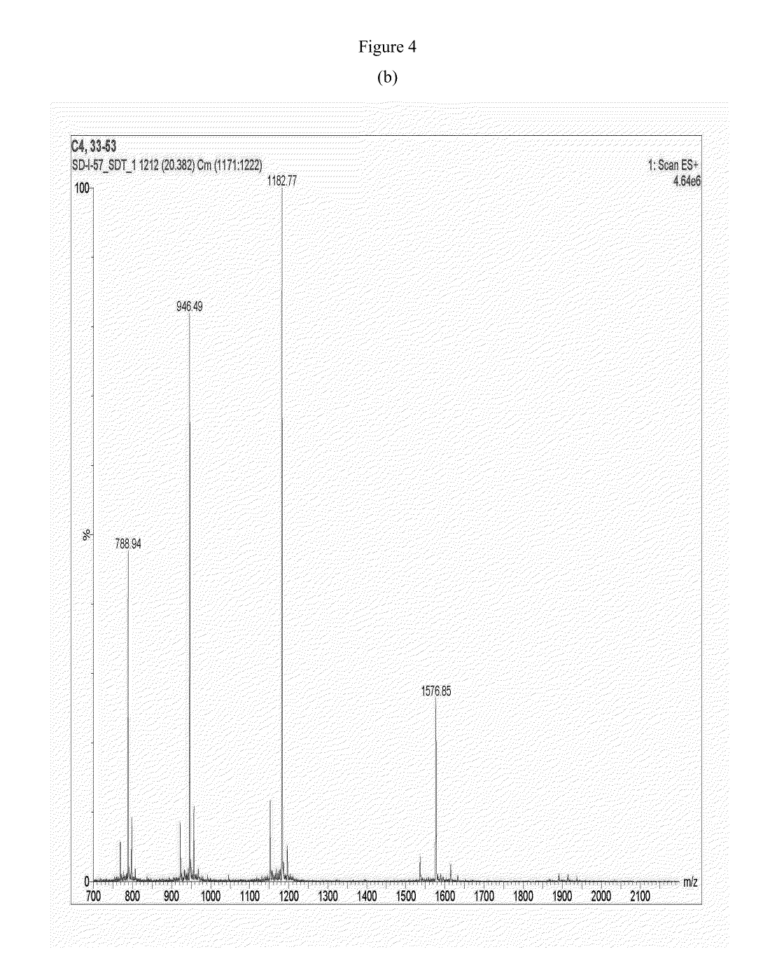

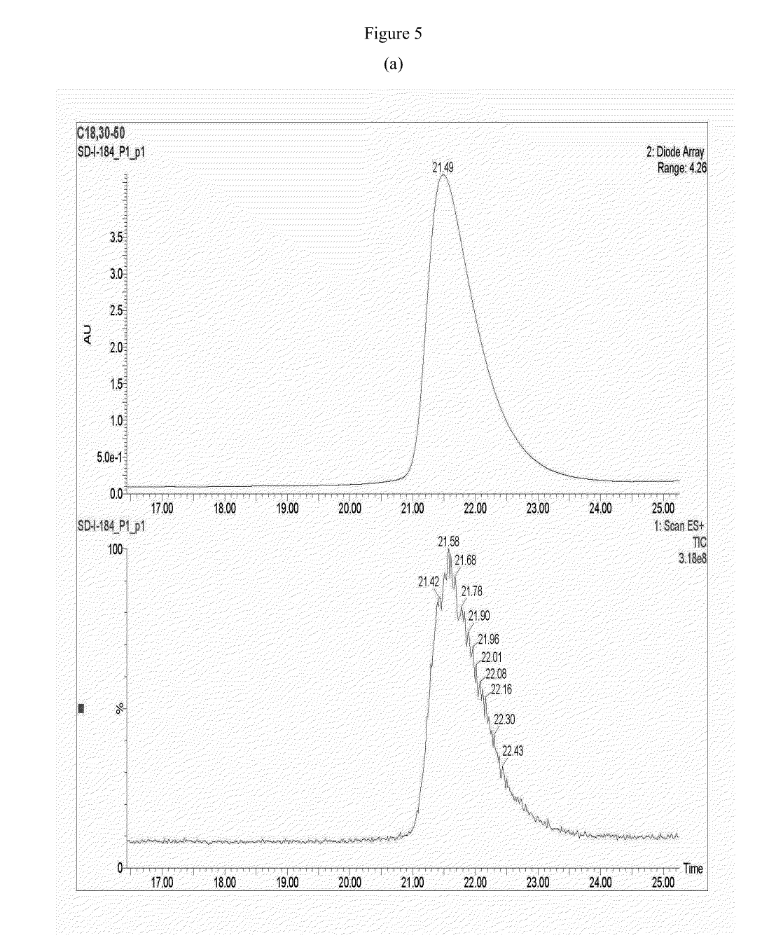

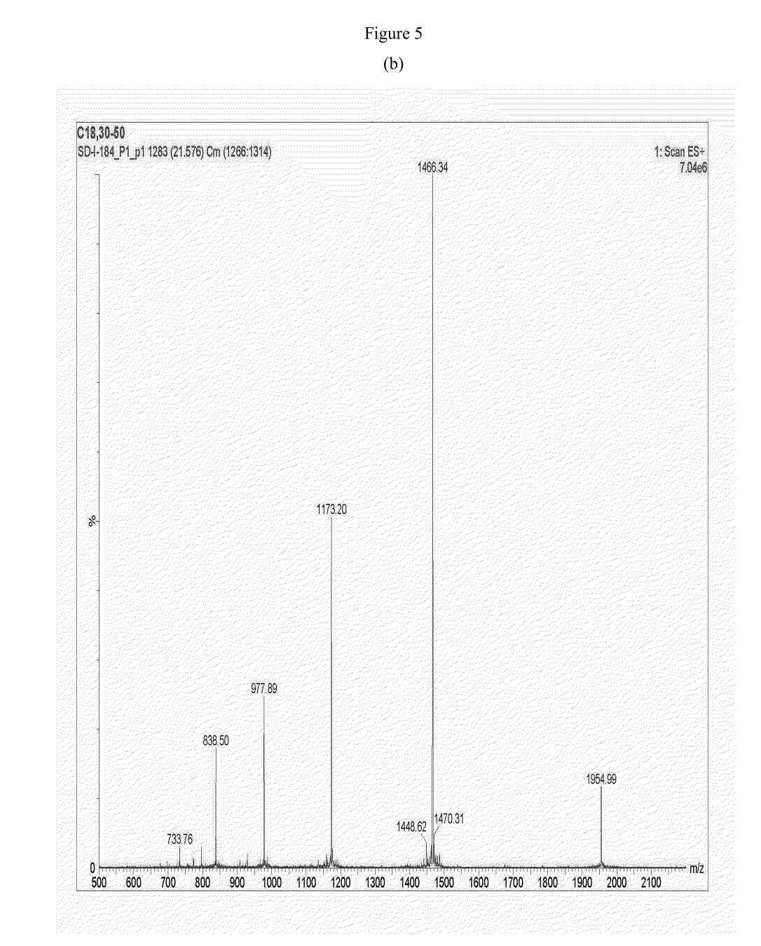

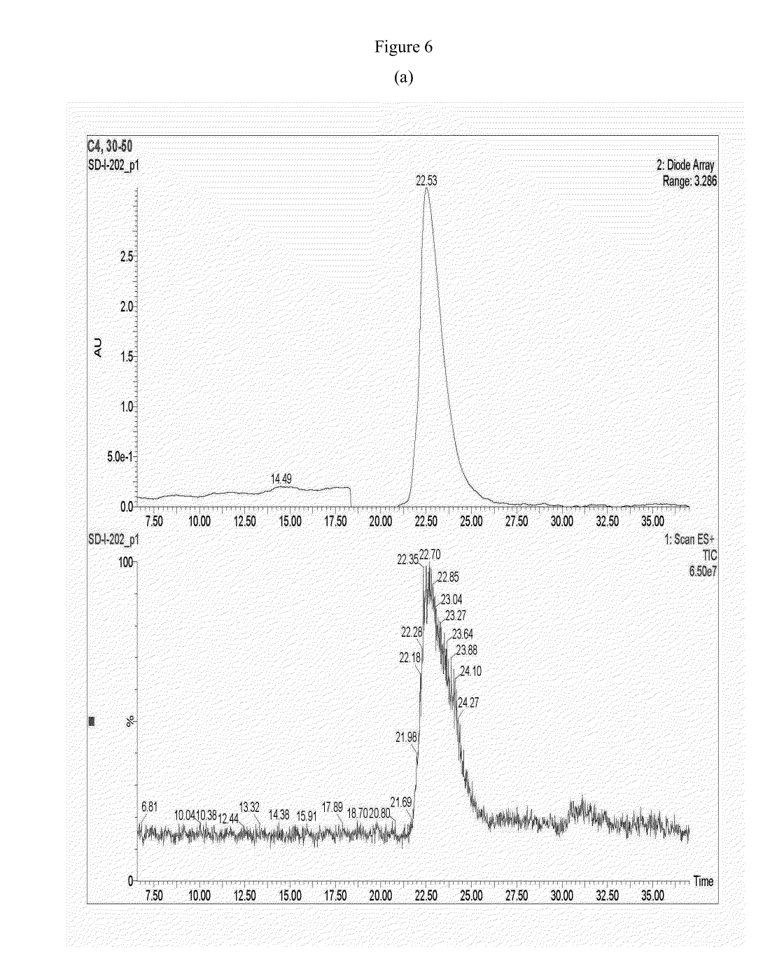

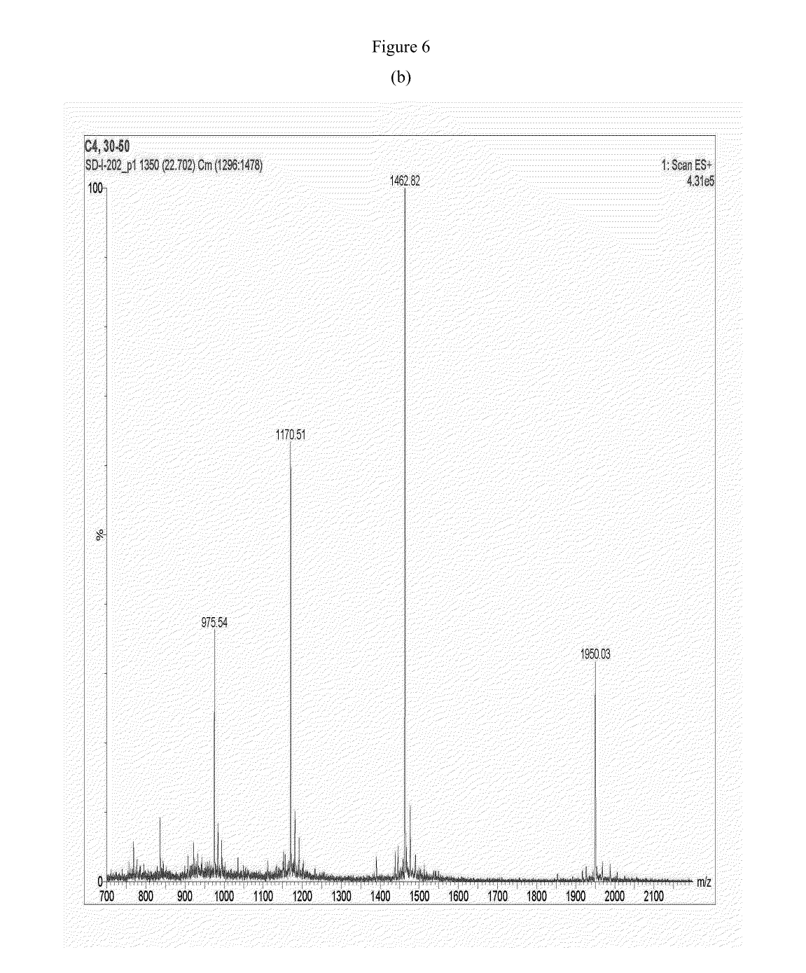

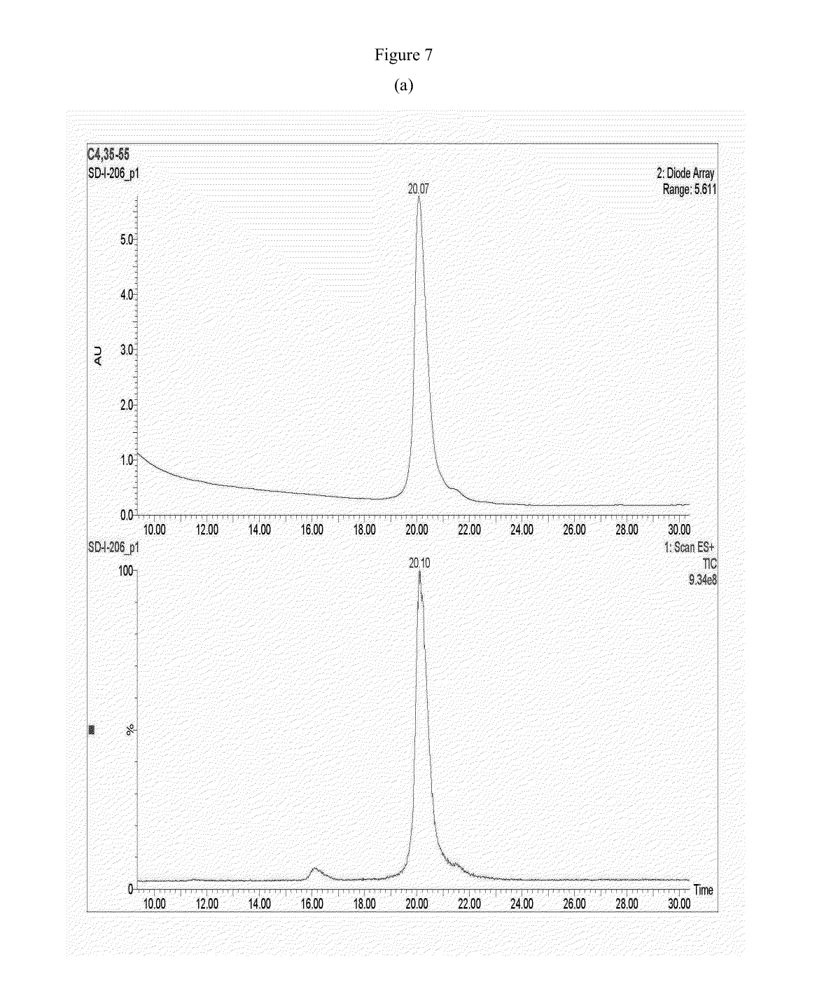

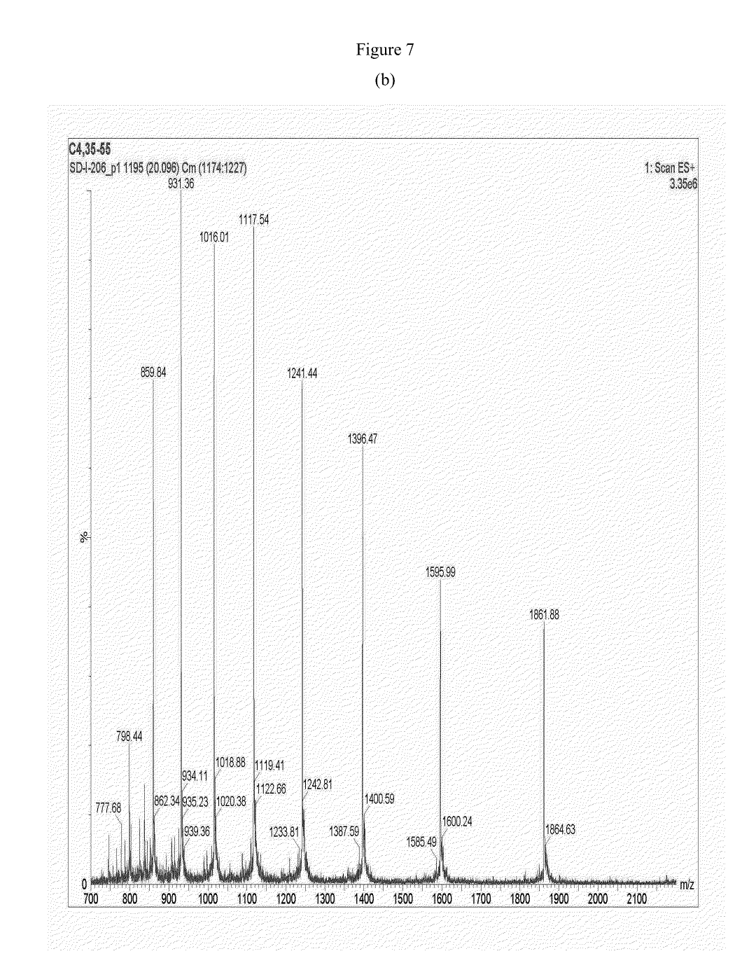

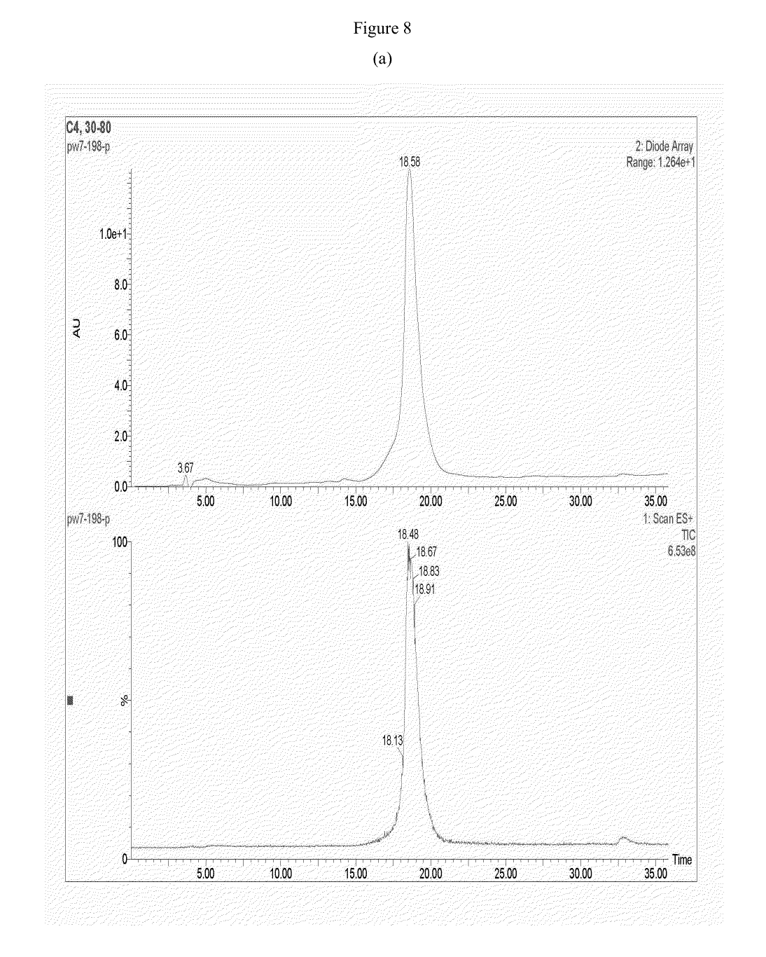

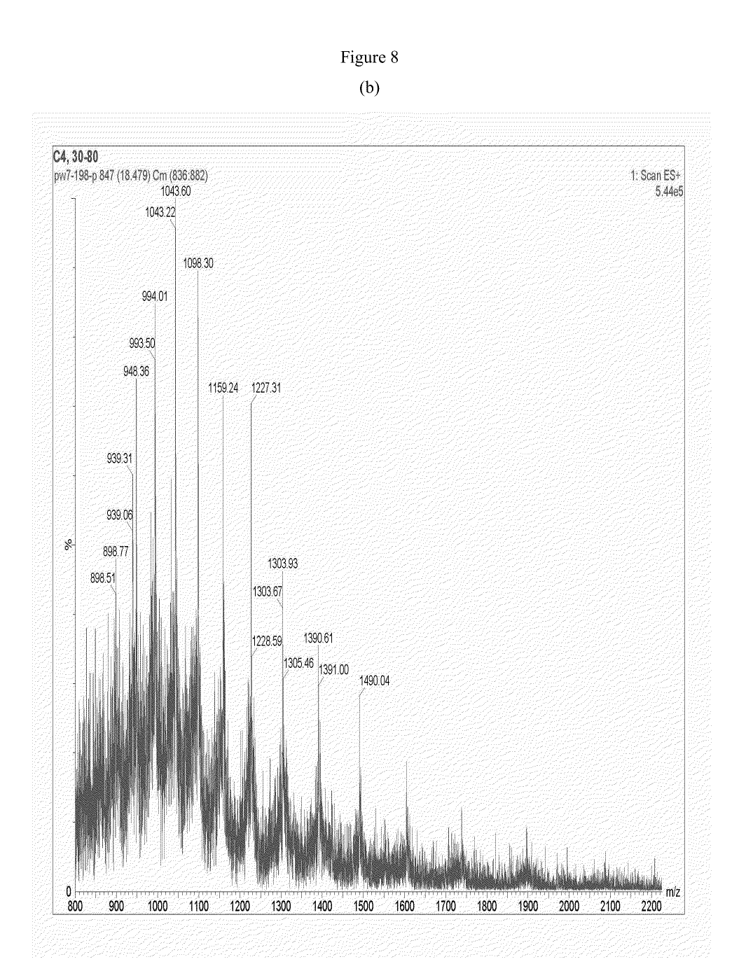

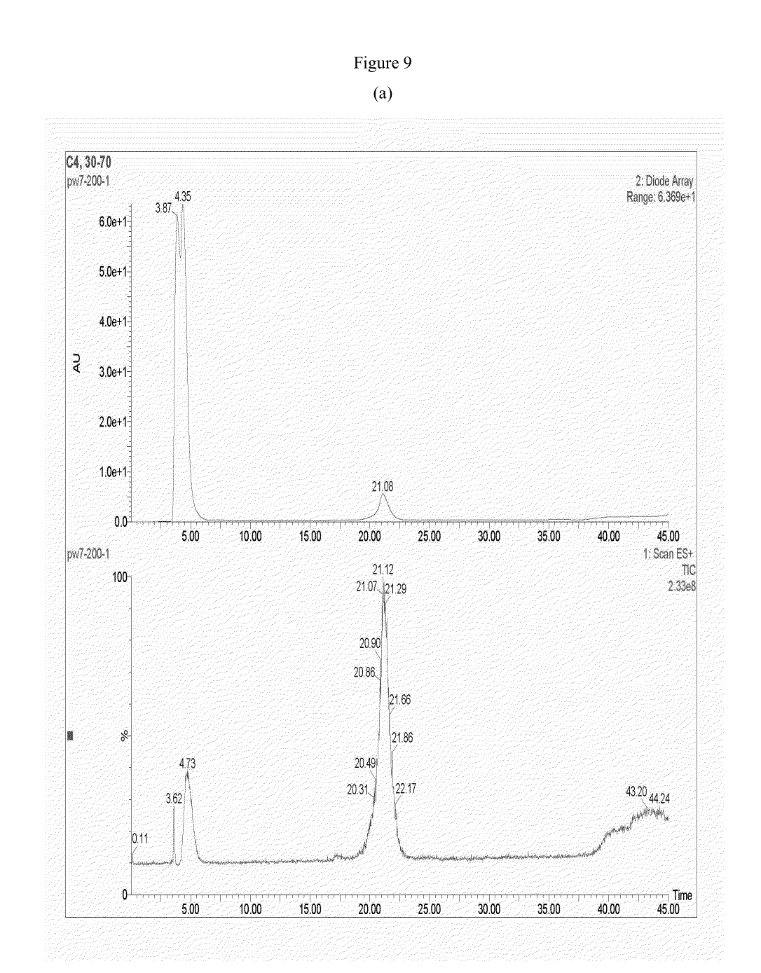

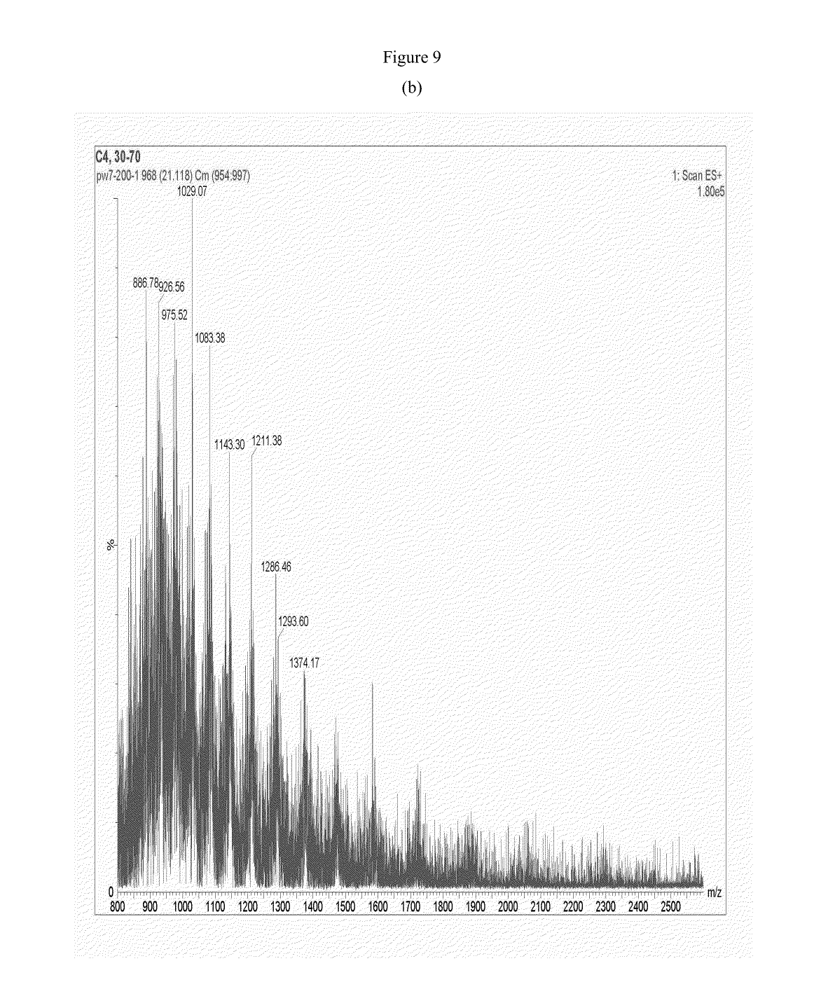

As shown in FIG. 1, experimental data indicated that significant EPO activity was detected at the concentration of <1.0 ng/ml with synthetic sample #100-8. Sterilization of #100.8 by 0.22 μM Millipore filtration (#100.8/0.22 μM) significantly reduced activity while sterilization with radiation did not (#100.8/10,000 Rad). PW8-100 and PW8-103 (alternative folding conditions) had significantly less activity than #100.8 and almost all activity was removed by 0.22 μM filtration (PW8-100/0.22 μM and PW8-103/0.22 μM).

### 3. Synthesis of EPO-2—General Procedure

### 3.1 Solid Phase Peptide Synthesis Using Fmoc-Strategy

Automated peptide synthesis was performed on an Applied Biosystems Pioneer continuous flow peptide synthesizer. Peptides were synthesized under standard automated Fmoc protocols. The deblock mixture was a mixture of 100:2:2 of DMF/piperidine/DBU. The following Fmoc amino acids and pseudoproline dipeptides from Novabiochem® were employed: Fmoc-Ala-OH, Fmoc-Arg(Pbf)-OH, Fmoc-Asn(Trt)-OH, Fmoc-Asp(OtBu)-OH, Boc-Thz-OH, Fmoc-Glu(OtBu)-OH, Fmoc-Gln(Trt)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Val-OH, Fmoc-Asp(OtBu)-Thr(ψ^{Me,Me}Pro)-OH, Fmoc-Ile-Ser (ψ^{Me,Me}Pro)-OH, Fmoc-Leu-Thr(ψ^{Me,Me}Pro)-OH, Fmoc-Ser(tBu)-Ser(ψ^{Me,Me}Pro)-OH, Fmoc-Tyr(tBu)-Ser(ψ^{Me,Me}Pro)-OH, Fmoc-Tyr(tBu)-Thr(ψ^{Me,Me}Pro)-OH, Fmoc-Val-Ser(ψ^{Me,Me}Pro)-0H.

Upon completion of the automated synthesis on a 0.05 mmol scale, the peptide resin was washed into a peptide cleavage vessel with DCM. The resin cleavage was performed with TFA/H₂O/triisopropylsilane (95:2.5:2.5 v/v) solution or DCM/AcOH/TFE (8:1:1 v/v) for 45 min (x2). The liquid was blown off with nitrogen. The oily residue was extracted with diethyl ether and centrifuged to give a white pellet. After the ether was decanted, the solid was lyophilized or purified for further use.

### 3.2 Preparation of Peptidyl Esters

The fully protected peptidyl acid (1.0 equiv) cleaved from resin using DCM/TFE/AcOH (8:2:2, v/v), and the amino acid ester hydrochloride (3.0 equiv) were dissolved in CHCl₃/TFE (3:1) and cooled to −10° C. HOOBt (3.0 equiv) and EDCI (3.0 equiv) were then added. The reaction mixture was stirred at room temperature for 4 h. The solvent was gently blown off by a nitrogen stream and the residue was washed with H₂O/AcOH (95:5, v/v). After centrifugation, the pellet was dissolved in TFA/H₂O/TIS (95:2.5:2.5) and stirred at room temperature for 1 h. The solvent was removed and the residue was triturated with cold ether. The resulting solid was dissolved in MeCN/H₂O/AcOH (47.5:47.5:5, v/v) for further analysis and purification.

3.3 Native Chemical Ligation with Peptidyl 2-(ethyldithio)phenol Ester

N-terminal peptide ester (1.5 equiv) and C-terminal peptide (1.0 equiv) were dissolved in ligation buffer (6 M Gdn.HCl, 100 mM Na₂HPO₄, 50 mM TCEP.HCl, pH 7.2˜7.3). The resulting solution was stirred at room temperature, and monitored using LC-MS. The reaction was quenched with MeCN/H₂O/AcOH (47.5:47.5:5) and purified by HPLC.

3.4 Native Chemical Ligation with Peptidyl Alkylthio Ester

N-terminal peptide ester (1.5 equiv) and C-terminal peptide (1.0 equiv) were dissolved in ligation buffer (6 M Gdn.HCl, 300 mM Na₂HPO₄, 20 mM TCEP.HCl, 200 mM 4-mercaptophenylacetic acid (MPAA), pH 7.2˜7.3). The resulting solution was stirred at room temperature, and monitored using LC-MS. The reaction was quenched with MeCN/H₂O/AcOH (47.5:47.5:5) and purified by HPLC.

### 3.5 Metal-Free Dethiylation

To a solution of the purified ligation product in 0.2 ml of degassed buffer (6 M Gdn.HCl, 200 mM NaH₂PO₄) was added 0.2 ml of 0.5 M bond-breaker® TCEP solution (Pierce), 0.05 ml of 2-methyl-2-propanethiol and 0.1 ml of radical initiator VA-044 (0.1 M in H₂O). The reaction mixture was stirred at 37° C. and monitored by LC-MS. Upon completion, the reaction was quenched by the addition of MeCN/H₂O/AcOH (47.5:47.5:5) and further purified by HPLC.

### 4. Preparation and Characterization of Glycopeptides.

Glycopeptide 4:

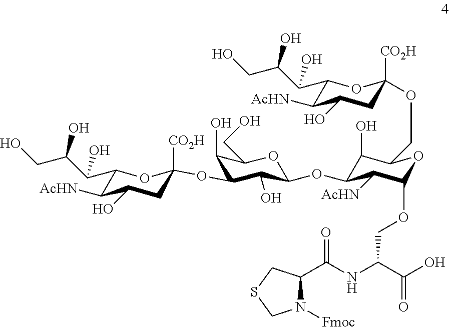

Fully protected glycophorin cassette (20 mg) (Schwarz, J. B.; Kuduk, S. D.; Chen, X.-T.; Sames, D.; Glunz, P. W.; Danishefsky, S. J. J. Am. Chem. Soc. 1999, 121, 2662-2673) was dissolved in 0.75 mL of MeOH. The resulting solution was carefully added 0.5 mL of 1 N NaOH solution dropwise, and stirred at rt for 3 h. The reaction was cooled to 0° C., and quenched by slow addition of 380 μL of 1 N HCl. The resulting mixture was concentrated, and dried upon lyophilization. The above obtained material was mixed with Fmoc-Thz-OSu (16 mg, 2.5 equiv) in 200 μL of dimethoxyethane (DME) and 200 μL of DMF. To the resulting mixture was added 200 μL of Na₂CO₃ solution (110 mg in 1 mL of water), and the reaction was stirred at rt for 1 h. The reaction was quenched with CH₃CN/H₂O/AcOH (30:65:5), and purified using RP-HPLC (linear gradient 18-38% solvent B over 30 min, Microsorb 300-5 C18 column, 16 mL/min, 230 nm). Product eluted at 19-21 min. The fractions were collected, and concentrated via lyophilization to provide peptide 4 (6.6 mg, 43%) as a white solid.

Glycopeptide 4: Calcd for C₅₈H₇₉N₅O₃₂S: 1390.33 Da(average isotopes), [M+2H]²⁺ m/z=696.16; observed: [M+H]⁺ m/z=1392.0, [M+2H]²⁺ m/z=696.1.

Glycopeptide 6:

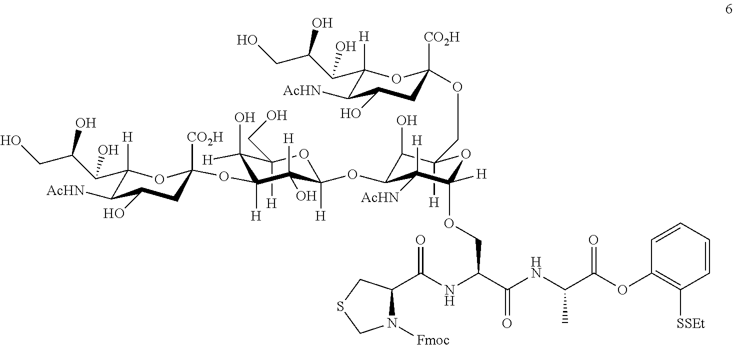

Glycopeptide 6: Calcd for C₆₉H₉₂N₆O₃₃S₃: 1629.28 Da(average isotopes); observed: [M+H]⁺ m/z=1630.81.

### Peptide 7:

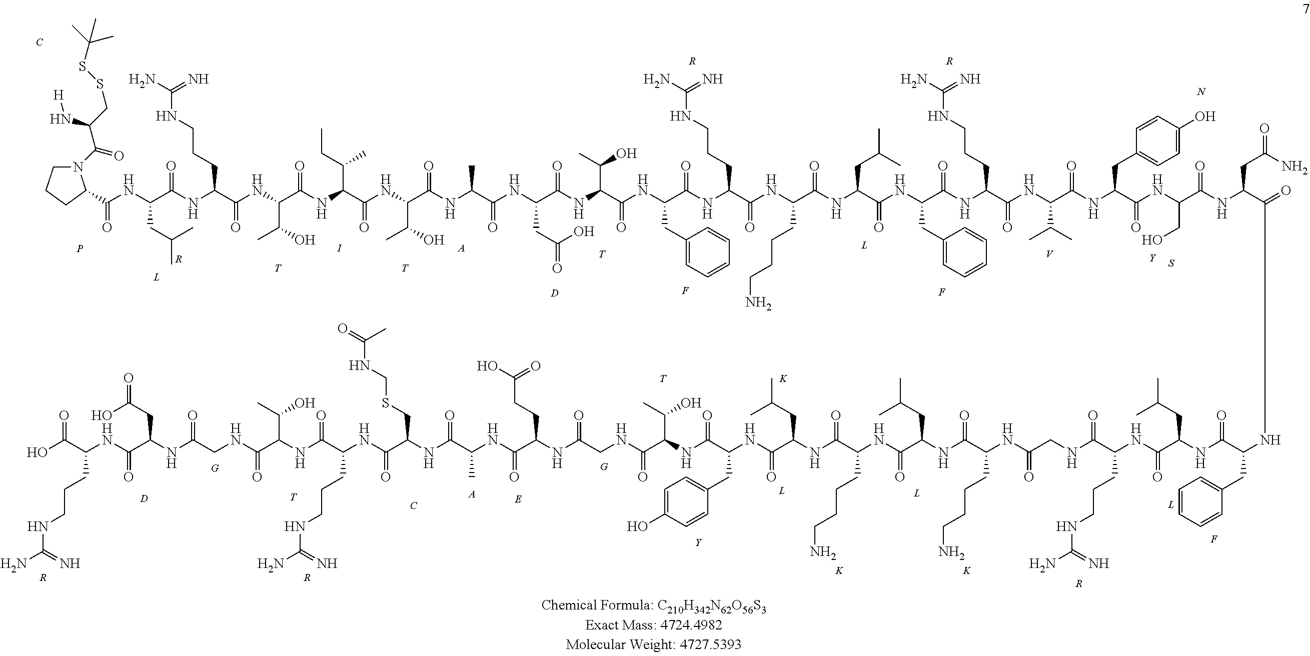

Peptide 7 was prepared according to General Procedure A for SPPS using Fmoc-Arg(Pbf)-Nova Syn® TGT resin, Fmoc-Cys(Acm)-OH, Boc-Cys(StBu)-OH, pseudoproline dipeptides Fmoc-Asp(OtBu)-Thr(ψ^{Me,Me}Pro)-OH, Fmoc-Ile-Ser(ψ^{Me,Me}Pro)-OH, Fmoc-Leu-Thr(ψ^{Me,Me}Pro)-OH, Fmoc-Tyr(tBu)-Ser(ψ^{Me,Me}Pro)-OH, Fmoc-Tyr(tBu)-Thr(ψ^{Me,Me}Pro)-OH, and other standard Fmoc amino acids from Novabiochem®. After cleavage and global deprotection using the TFA/TIS/H₂O protocol, the crude material was further purified using RP-HPLC (linear gradient 27-47% solvent B over 30 min, Microsorb 300-5 C4 column, 16 mL/min, 230 nm). Product eluted at 19-21 min. The fractions were collected, and concentrated via lyophilization to provide peptide 7 (42.5 mg, 18%) as a white solid.

Glycopeptide 7: Calcd for C₂₁₀H₃₄₂N₆₂O₅₆S₃: 4727.54 Da(average isotopes), [M+3H]³⁺ m/z=1576.85, [M+4H]⁴⁺ m/z=1182.89, [M+5H]⁵⁺ m/z=946.51, [M+6H]⁶⁺ m/z=788.92; observed: [M+3H]³⁺ m/z=1576.85, [M+4H]⁴⁺ m/z=1182.77, [M+5H]⁵⁺ m/z=946.49, [M+6H]⁶⁺ m/z=788.94.

Glycopeptide 8:

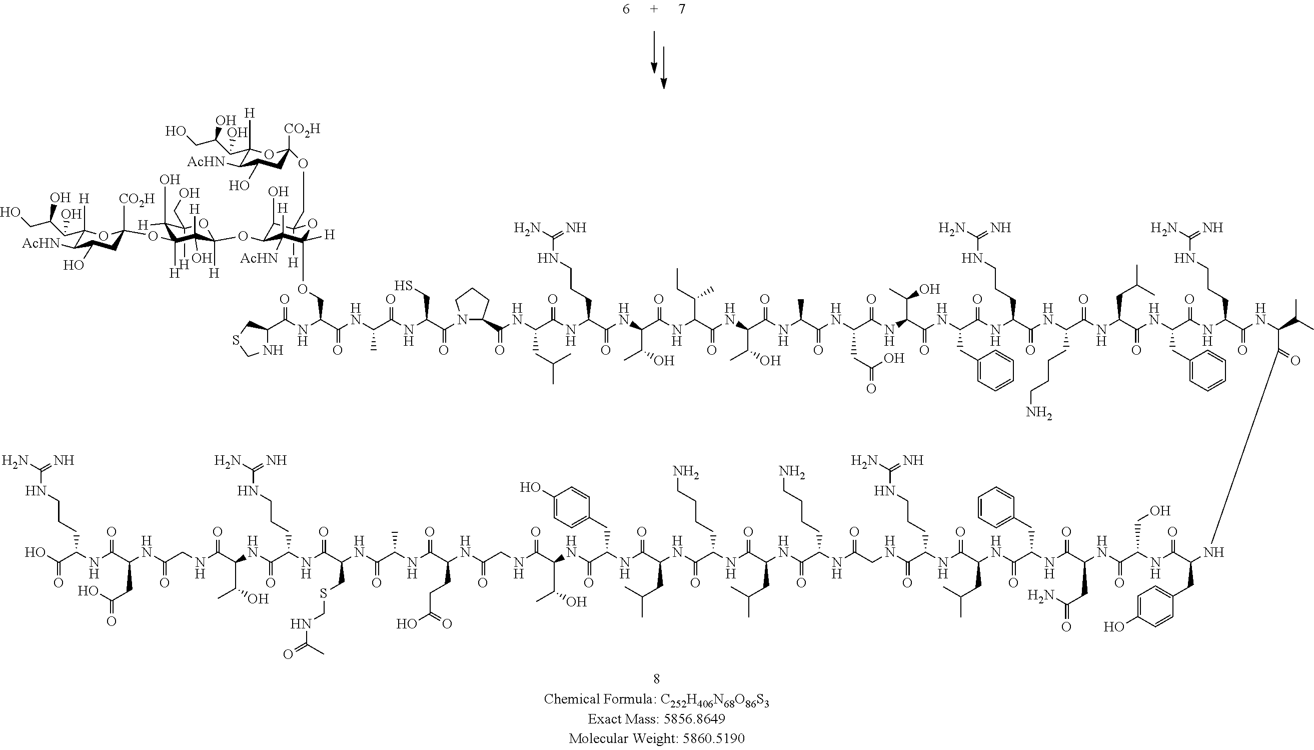

According to General Procedure C, peptide 6 (1.58 mg, 0.97 μmol, 1.0 equiv) and peptide 7 (5.0 mg, 1.07 μmol, 1.1 equiv) were dissolved in 250 μL of NCL buffer under an argon atmosphere. The resulting mixture was stirred at room temperature and the reaction was monitored by LC-MS . After 2 h, the reaction was diluted with 2 mL of CH₃CN/H₂O (1:1), and concentrated via lyophilization. To the resulting residue was added 150 μL of DMSO followed by the addition of 20 μL of piperidine. The slurry was stirred at rt for 10 min and quenched with 2 mL of CH₃CN/H₂O/AcOH (24:71:5) and 100 μL of Bond-Breaker® TCEP solution, and then purified directly by RP-HPLC (linear gradient 26-46% solvent B over 30 min, Microsorb 300-5 C4 column, 16 mL/min, 230 nm). Product eluted at 21-22.5 min. The fractions were collected, and concentrated via lyophilization to afford 3.1 mg ligated peptide 8 (55%, two steps) as a white solid.

Glycopeptide 8: Calcd for C₂₅₂H₄₀₆N₆₈O₈₆S₃: 5860.52 Da(average isotopes), [M+3H]³⁺ m/z=1954.51, [M+4H]⁴⁺ m/z=1466.13, [M+5H]⁵⁺ m/z=1173.10, [M+6H]⁶⁺ m/z=977.75, [M+7H]⁷⁺ m/z=838.22; observed: [M+3H]³⁺ m/z=1954.99, [M+4H]⁴⁺ m/z=1466.34, [M+5H]⁵⁺ m/z=1173.20, [M+6H]⁶⁺ m/z=977.89, [M+7H]⁷⁺ m/z=838.50.

Glycopeptide 9:

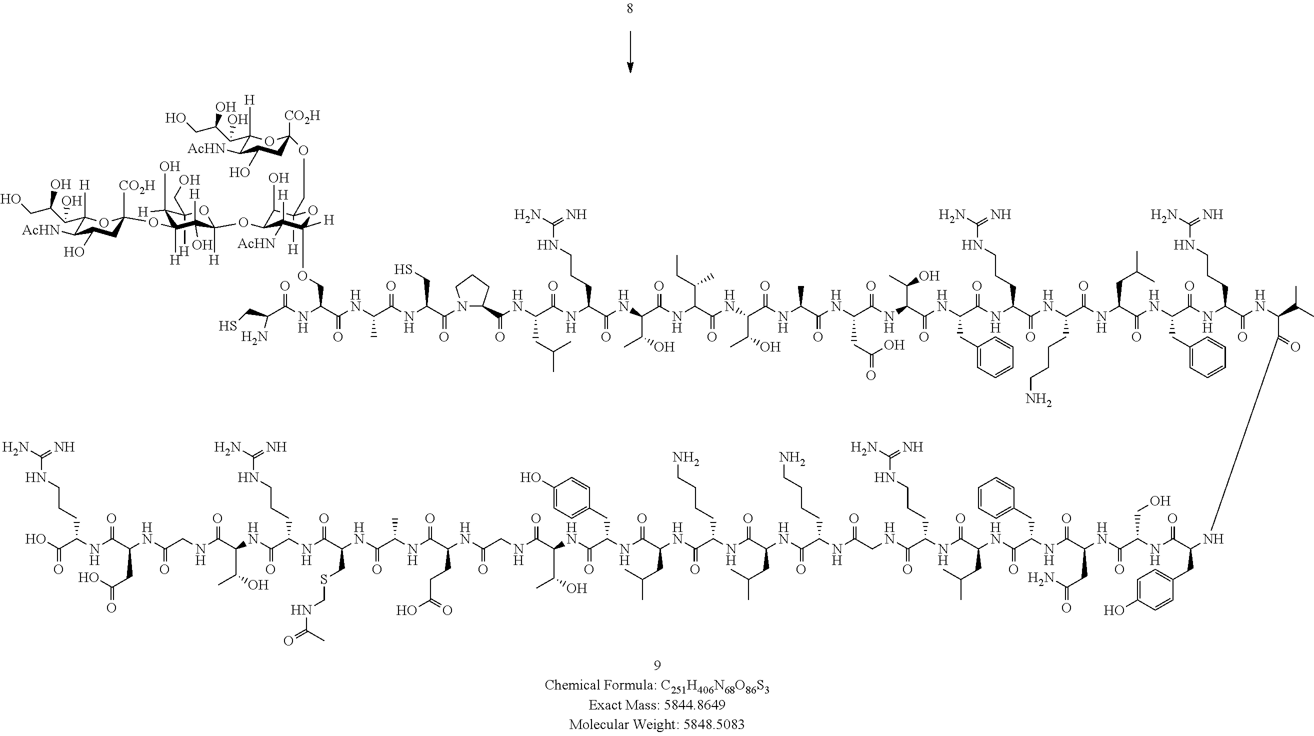

Glycopeptide 8 (5.5 mg, 0.94 μmol) was dissolved in 400 μL of buffer (6 M Gdn.HCl, 100 mM Na₂HPO₄, 50 mM TCEP.HCl, pH 6.5) under an argon atmosphere. To the solution was added MeONH₂.HCl (30 mg) in one portion. The resulting mixture was stirred at rt and the reaction was monitored by LC-MS. After 3 h, the reaction was diluted with 3 mL of CH₃CN/H₂O/AcOH (30:65:5) and 100 μL of Bond-Breaker® TCEP solution, then purified directly by RP-HPLC (linear gradient 30-50% solvent B over 30 min, Microsorb 300-5 C4 column, 16 mL/min, 230 nm). Product eluted at 19-21 min. The fractions were collected, and concentrated via lyophilization to afford 4.7 mg ligated peptide 9 (86%) as a white solid.

Glycopeptide 9: Calcd for C₂₅₁H₄₀₆N₆₈O₈₆S₃: 5848.51 Da(average isotopes), [M+3H]³⁺ m/z=1950.50, [M+4H]⁴⁺ m/z=1463.13, [M+5H]⁵⁺ m/z=1170.70, [M+6H]⁶⁺ m/z=975.75; observed: [M+3H]³⁺ m/z=1950.03, [M+4H]⁴⁺ m/z=1462.82, [M+5H]⁵⁺ m/z=1170.51, [M+6H]⁶⁺ m/z=975.54.

Glycopeptide 14:

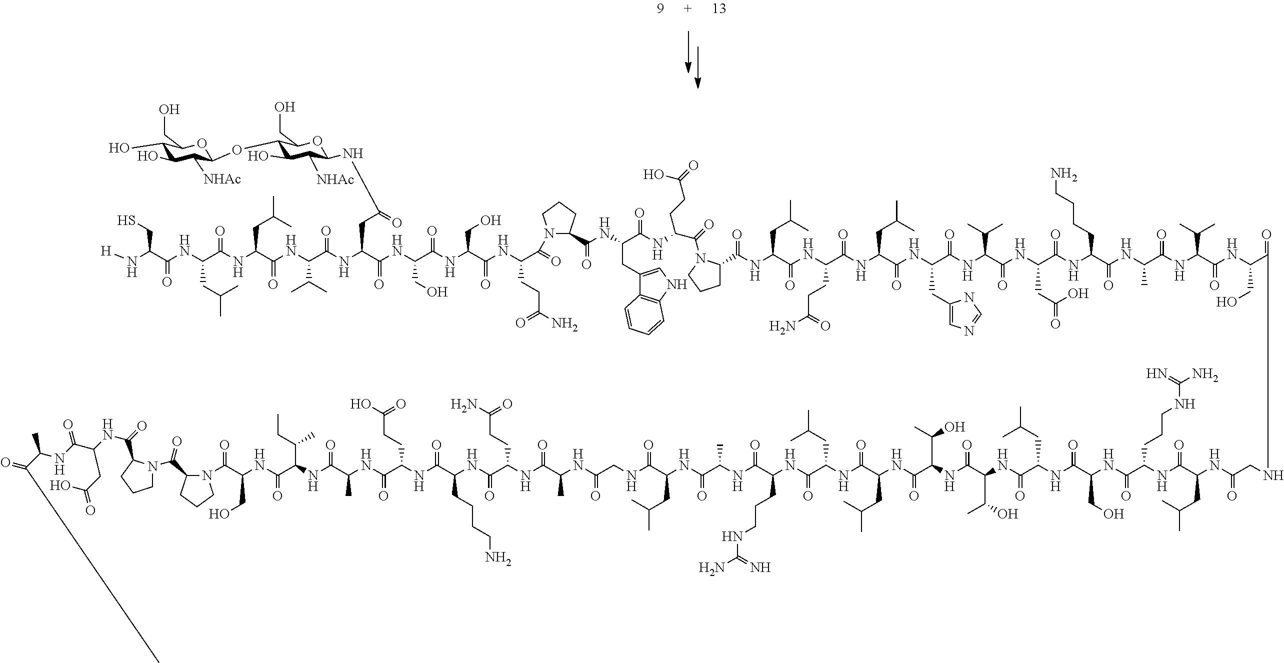

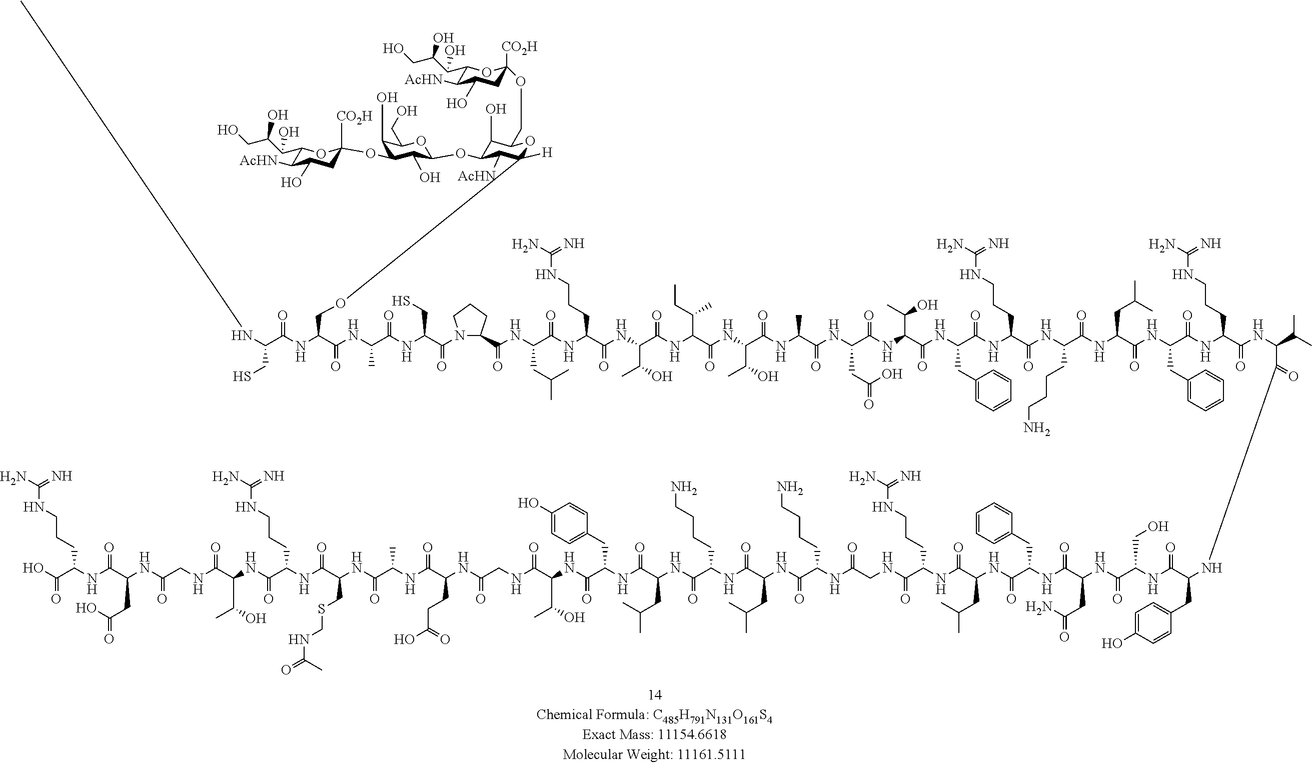

According to General Procedure D, glycopeptides 9 (2.46 mg, 0.45 mmol, 1.03 equiv) and 13 (2.55 mg, 0.44 mmol, 1.00 equiv) were dissolved in 200 μL of NCL buffer under an argon atmosphere. The resulting mixture was stirred at room temperature and the reaction was monitored by LC-MS. After 18 h, to the reaction was added 15 mg of MeONH₂.HCl and 3 mg of DTT in one portion. The resulting mixture was further stirred at rt for 3 h under Ar. The reaction was quenched with 3 mL of CH₃CN/H₂O/AcOH (30:65:5) and 100 μL of Bond-Breaker® TCEP solution, and then purified directly by RP-HPLC (linear gradient 28-48° A solvent B over 30 min, Microsorb 300-5 C4 column, 16 mL/min, 230 nm). Product eluted at 20-22 min. The fractions were collected, and concentrated via lyophilization to afford 3.51 mg ligated peptide 14 (72%, two steps) as a white solid.

Glycopeptide 14: Calcd for C₄₈₅H₇₉₁N₁₃₁O₁₆₁S₄: 11161.51 Da(average isotopes), [M+6H]⁶⁺ m/z=1861.25, [M+7H]⁷⁺ m/z=1595.50, [M+8H]⁸⁺ m/z=1396.19, [M+9H]⁹⁺ m/z=1241.17, [M+10H]¹⁰⁺ m/z=1117.15, [M+11H]¹¹⁺ m/z=1015.68, [M+12H]¹²⁺ m/z=931.13, [M+13H]¹³⁺ m/z=859.58, [M+14H]¹⁴⁺ m/z=798.25; observed: [M+6H]⁶⁺ m/z=1861.88, [M+7H]⁷⁺ m/z=1595.99, [M+8H]⁸⁺ m/z=1396.47, [M+9H]⁹⁺ m/z=1241.44, [M+10H]¹⁰⁺ m/z=1117.54, [M+11H]¹¹⁺ m/z=1016.01, [M+12H]¹²⁺ m/z=931.36, [M+13H]¹³⁺ m/z=859.84, [M+14H]¹⁴⁺ m/z=798.44.

Procedure for one pot NCL followed by dethiofulrization: Glycopeptides 17 (1.95 mg, 1.20 equiv) and 15 (2.53 mg, 1.00 equiv) were dissolved in 150 μL of NCL buffer (6 M GND/HCl, 0.1 M Na₂HPO₄, 50 mM TCEP, pH 7.0) under an argon atmosphere. The resulting mixture was stirred at room temperature. After 4 h, to the reaction was added 180 ul NCL buffer (6 M GND/HCl, 0.1 M Na₂HPO₄, 50 mM TCEP, 0.3 M MPAA, pH 7.0) and glycopeptides 18 (3.20 mg, 0.7 equiv) in one portion. The resulting mixture was further stirred at rt for 12 h under Ar. The reaction was quenched 3 mL (6 M GND/HCl, 0.1 M Na₂HPO₄) and 50 μL of Bond-Breaker® TCEP solution, and then concentrated by ultrafiltration (mwco 10,000) to 300 uL. Repeat twice to remove materials of low molecular weight.

The mixture was dissolved in 2 mL buffer (5.6 M GND/HCl, 0.1 M Na₂HPO₄, 0.3 M TCEP, pH 6.8) under an argon atmosphere, followed by addition of 60 uL t-BuSH and VA-044 (90 ul, 0.1 M in water). The resulting mixture was stirred at 37° C. for 12 h. The reaction was quenched 5 mL (6 M GND/HCl, 0.1 M Na₂HPO₄) and purified directly by RP-HPLC (linear gradient 40-60% solvent B over 30 min, Microsorb 300-5 C4 column, 16 mL/min, 230 nm). Product eluted at 20-22 min. The fractions were collected, and concentrated via lyophilization to afford 3.17 mg ligated peptide 23 (54%, three steps) as a white solid.

Glycopeptide 23: Calcd for C₉₁₁H₁₄₇₂N₂₄₆O₃₀₁S₅: 20834.60 Da(average isotopes), [M+14H]¹⁴⁺ m/z=1489.19, [M+15H]¹⁵⁺ m/z=1389.97, [M+16H]¹⁶⁺ m/z=1303.16, [M+17H]¹⁷⁺ m/z=1226.56, [M+18H]¹⁸⁺ m/z=1158.48, [M+19H]¹⁹⁺ m/z=1097.56, [M+20H]²⁰⁺ m/z=1042.73, [M+21H]²¹⁺ m/z=993.12, [M+22H]²²⁺ m/z=948.02; observed [M+14H]¹⁴⁺ m/z=1490.04, [M+15H]¹⁵⁺ m/z=1390.61, [M+16H]¹⁶⁺ m/z=1303.93, [M+17H]¹⁷⁺ m/z=1227.31, [M+18H]¹⁸⁺ m/z=1159.24, [M+19H]¹⁹⁺ m/z=1098.30, [M+20H]²⁰⁺ m/z=1043.60, [M+21H]²¹⁺ m/z=994.01, [M+22H]²²⁺ m/z=948.36.

Removal of Acm—Glycopeptide 1: 3.2 mg (0.15 μmol) glycopeptide 23 was dissolved in 1 mL degassed 70% AcOH/H₂O solution. To the above solution, 11 mg (0.066 mmol) AgOAc was added. After 6 hours, reaction was quenched by 2.5 mL solution of 1 M DTT in 6 M guanidine hydrochloride. White precipitate formed upon adding DTT solution. The mixture was stirred for 30 mins followed by centrifuge. The mixture was purified directly by RP-HPLC (linear gradient 40-60% solvent B over 30 min, Microsorb 300-5 C4 column, 16 mL/min, 230 nm). Product eluted at 20-22 min. The fractions were collected, and concentrated via lyophilization to afford 2.2 mg peptide 1 (70%) as a white solid. The peptide 1 was dissolved in 2.2 mL buffer (6 M GND/HCl, 20 mM DTT) to prevent aggregation and kept in −80° C.

Glycopeptide 1: Calcd for C₈₉₉H₁₄₅₂N₂₄₂O₂₉₇S₅: 20550.46 Da(average isotopes), [M+14H]¹⁴⁺ m/z=1468.89, [M+15H]¹⁵⁺ m/z=1371.03, [M+16H]¹⁶⁺ m/z=1285.41, [M+17H]¹⁷⁺ m/z=1209.85, [M+18H]¹⁸⁺ m/z=1142.69, [M+19H]¹⁹⁺ m/z=1082.61, [M+20H]²⁰⁺ m/z=1028.53; observed: [M+15H]¹⁵⁺ m/z=1374.17, [M+16H]¹⁶⁺ m/z=1286.46, [M+17H]¹⁷⁺ m/z=1211.38, [M+18H]¹⁸⁺ m/z=1143.30, [M+19H]¹⁹⁺ m/z=1083.38, [M+20H]²⁰⁺ m/z=1029.07.

Folding: The EPO peptide 1 above was diluted to 20.0 mL with 6 M GND/HCl in folding tube (mwco 10,000) and refolded by dialysis against 40 mM CuSO₄, 2% sarkosyl sodium (w/v), 50 mM Tris-HCl, pH 8.0. The mixture was concentrated to 3.0 mL by ultrafilter (mwco 10 kDa). The concentration of EPO was evaluated by UV at 280 nm. The EPO protein was stored at −80° C.

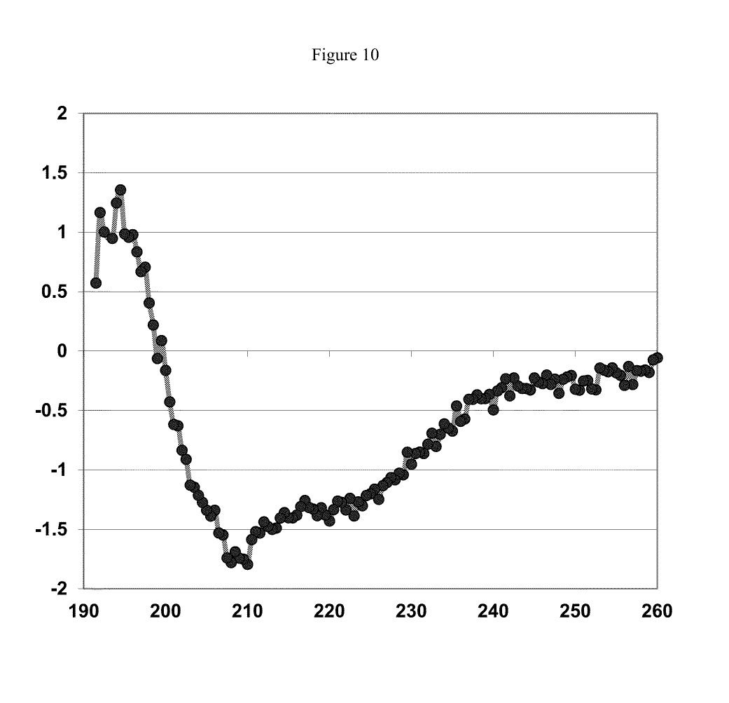

CD spectrum of fully synthetic, homogeneously glycosylated erythropoietin (chitoboise moieties at Asn²⁴, Asn³⁸ and Asn⁸³; and glycophorin at Ser¹²⁶) was depicted in FIG. 10.

### 4. Methods for EPO Bioassay

Tissue culture: An erythropoietin responsive human erythroleukemia cell line TF-1 (Kitamura, T.; Tange, T.; Terasawa, T.; Chiba, S.; Kuwaki, T.; Miyagawa, K.; Piao, Y F.; Miyazono, K.; Urabe, A.; Takaku, F. J. Cell Physiol. 1989, 140, 323-34), was obtained from the American Type Culture Collection (ATCC, Manassas, Va.) and maintained in IMDM medium containing 20% Serum Replacement (SR, Invitrogen, Grand Island, N.Y.), 80 mM 2-mercaptoethanol, 2 mM L-glutamine, 50 units/ml penicillin, 50 μg/ml streptomycin, 6 units/ml human recombinant erythropoietin [rhEPO (Procrit™), Johnson & Johnson, New Brunswick, N.J.]. TF-1 cells in log-phase expansion were harvested and evaluated for their proliferation and differentiation response to synthetic EPOs and clinical grade recombinant human EPO (epoetin alpha, Procrit™. Johnson & Johnson).

EPO Bioassay: 5,000 TF-1 cells/well/60 μl of IMDM medium containing 20% SR, 80 mM 2-mercaptoethanol, 2 mM L-glutamine, 50 units/ml penicillin, 50 μg/ml streptomycin in the presence or absence various doses of rhEPO or synthetic EPO was set up in a 384-wells plate in triplicates. After 72 hours culturing in a 5% CO₂ and humidified incubator, 6 μA of Alarma Blue (Invitrogen Inc. Grand Island, N.Y.) was added to each well and the cultures were incubated overnight. Fluorescence intensity of the culture in the 384-wells was measured using a Synergy H1 platereader (BioTek).

1. A composition of homogeneous, fully-glycosylated erythropoietin, wherein the primary amino acid sequence of the erythropoietin is as follows:
(SEQ ID NO: 1)
Ala-Pro-Pro-Arg-Leu-Ile-Cys-Asp-Ser-Arg-Val-Leu-Glu-Arg-Tyr-Leu-Leu-Glu-Ala-Lys-
Glu-Ala-Glu-Asn-Ile-Thr-Thr-Gly-Cys-Ala-Glu-His-Cys-Ser-Leu-Asn-Glu-Asn-Ile-Thr-
Val-Pro-Asp-Thr-Lys-Val-Asn-Phe-Tyr-Ala-Trp-Lys-Arg-Met-Glu-Val-Gly-Gln-Gln-Ala-
Val-Glu-Val-Trp-Gln-Gly-Leu-Ala-Leu-Leu-Ser-Glu-Ala-Val-Leu-Arg-Gly-Gln-Ala-Leu-
Leu-Val-Asn-Ser-Ser-Gln-Pro-Trp-Glu-Pro-Leu-Gln-Leu-His-Val-Asp-Lys-Ala-Val-Ser-
Gly-Leu-Arg-Ser-Leu-Thr-Thr-Leu-Leu-Arg-Ala-Leu-Gly-Ala-Gln-Lys-Glu-Ala-Ile-Ser-
Pro-Pro-Asp-Ala-Ala-Ser-Ala-Ala-Pro-Leu-Arg-Thr-Ile-Thr-Ala-Asp-Thr-Phe-Arg-Lys-
Leu-Phe-Arg-Val-Tyr-Ser-Asn-Phe-Leu-Arg-Gly-Lys-Leu-Lys-Leu-Tyr-Thr-Gly-Glu-Ala-
Cys-Arg-Thr-Gly-Asp-Arg,
or is SEQ ID NO: 1 having 1-10 amino acid substitutions, additions, and/or
deletions.

2. (canceled)

3. The composition of claim 1, wherein Arg¹⁶⁶ is deleted.

4. The composition of claim 1, wherein the primary amino acid sequence of the erythropoietin SEQ ID NO: 1 has 1-10 amino acid substitutions, addition, and/or deletions, wherein Asn²⁴, Asn³⁸, Asn⁸³ and Ser¹²⁶ are not mutated.

5. The composition of claim 1, wherein the erythropoietin has one or more disulfide bond formed between cysteine residues.

6. The composition of claim 5, wherein the erythropoietin has a disulfide bond formed between Cys⁷ and Cys¹⁶¹.

7. The composition of claim 1, wherein the erythropoietin is folded.

8. The composition of claim 1, wherein each of Asn²⁴, Asn³⁸ and Asn⁸³ is glycosylated with a glycan independently selected from:

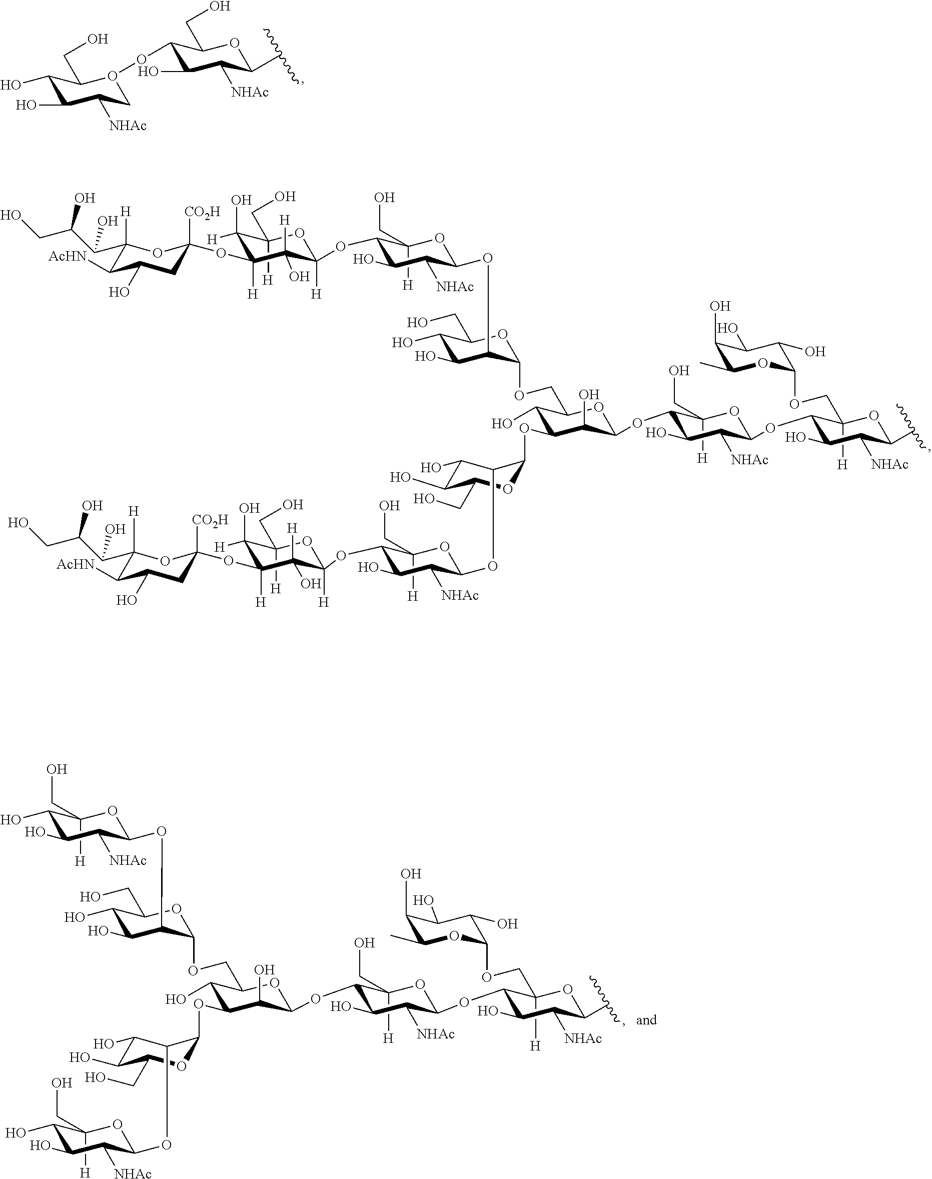

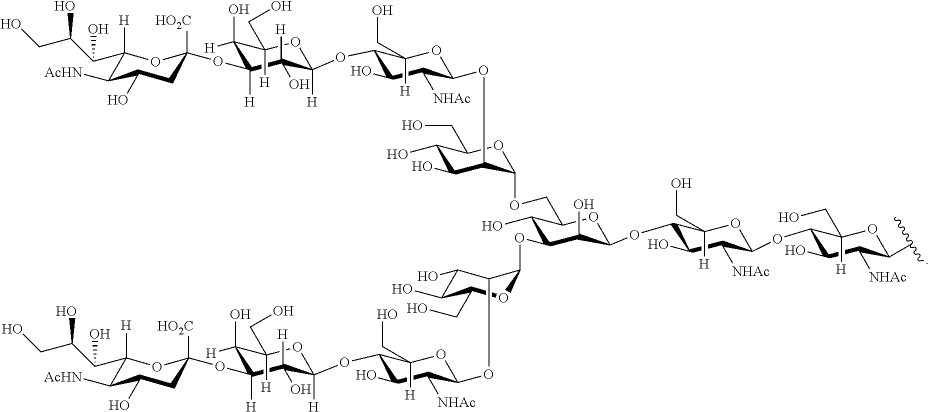

9. The composition of claim 1, wherein Asn²⁴, Asn³⁸ and Asn⁸³ are glycosylated with the same glycan selected from:

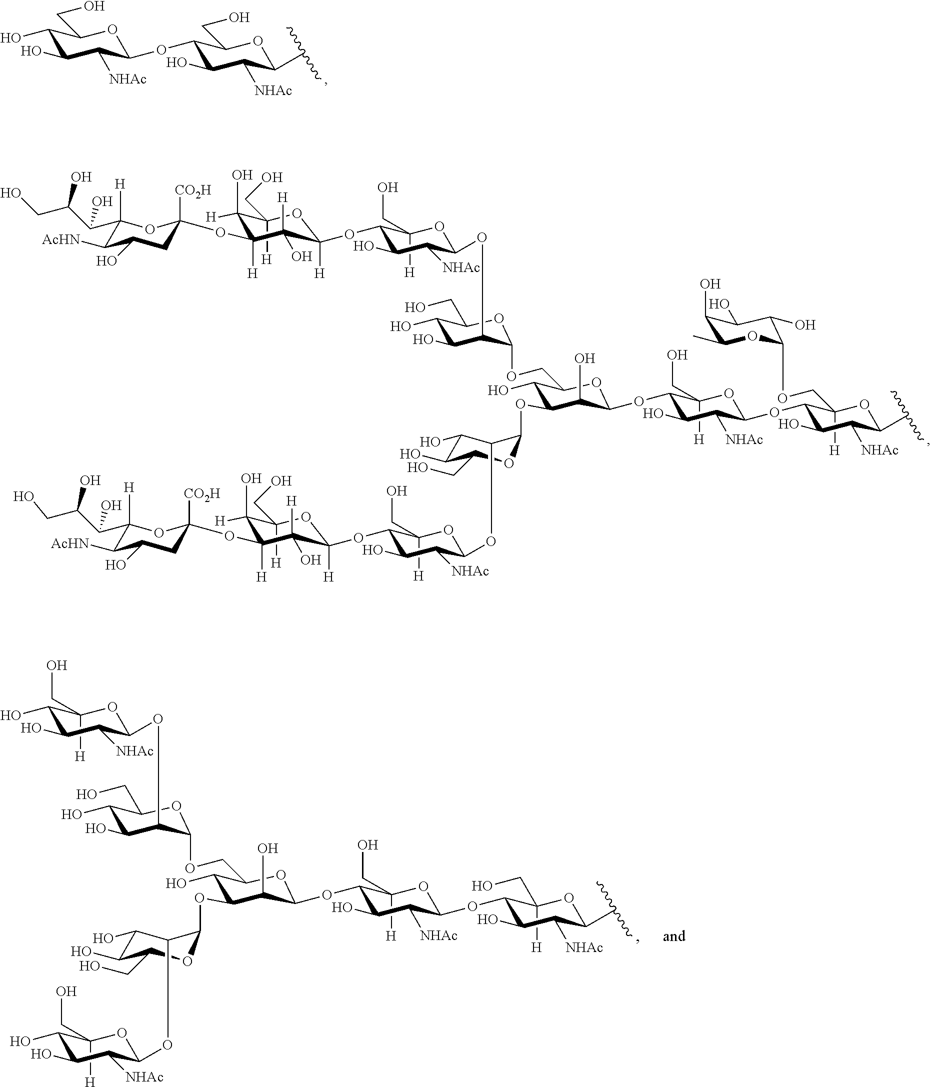

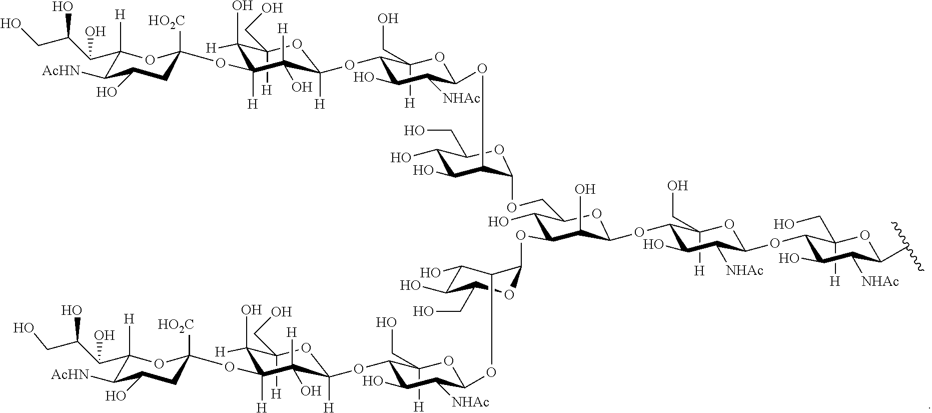

10. The composition of claim 8, wherein the glycan at Ser¹²⁶ is selected from

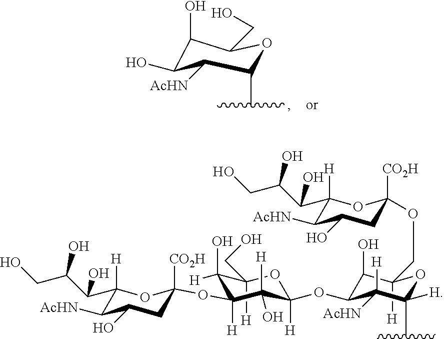

11-12. (canceled)

13. The composition of claim 1, wherein the erythropoietin has the following structure:

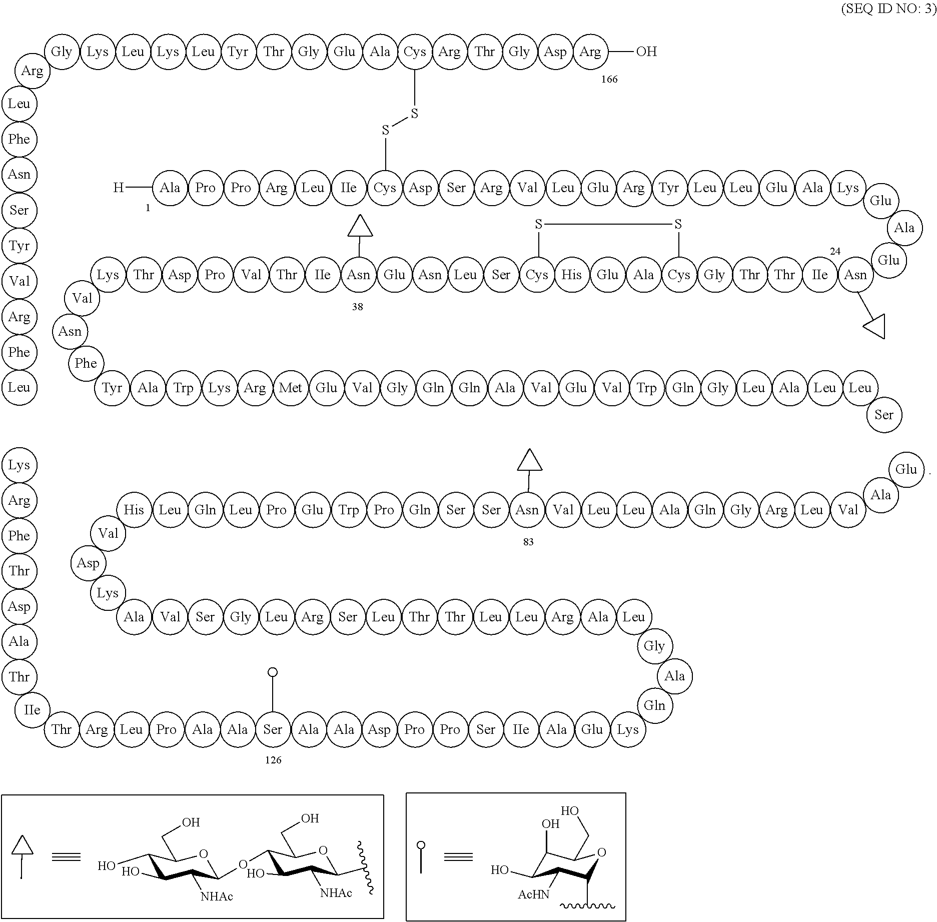

14. The composition of claim 1, wherein the erythropoietin has the following structure:

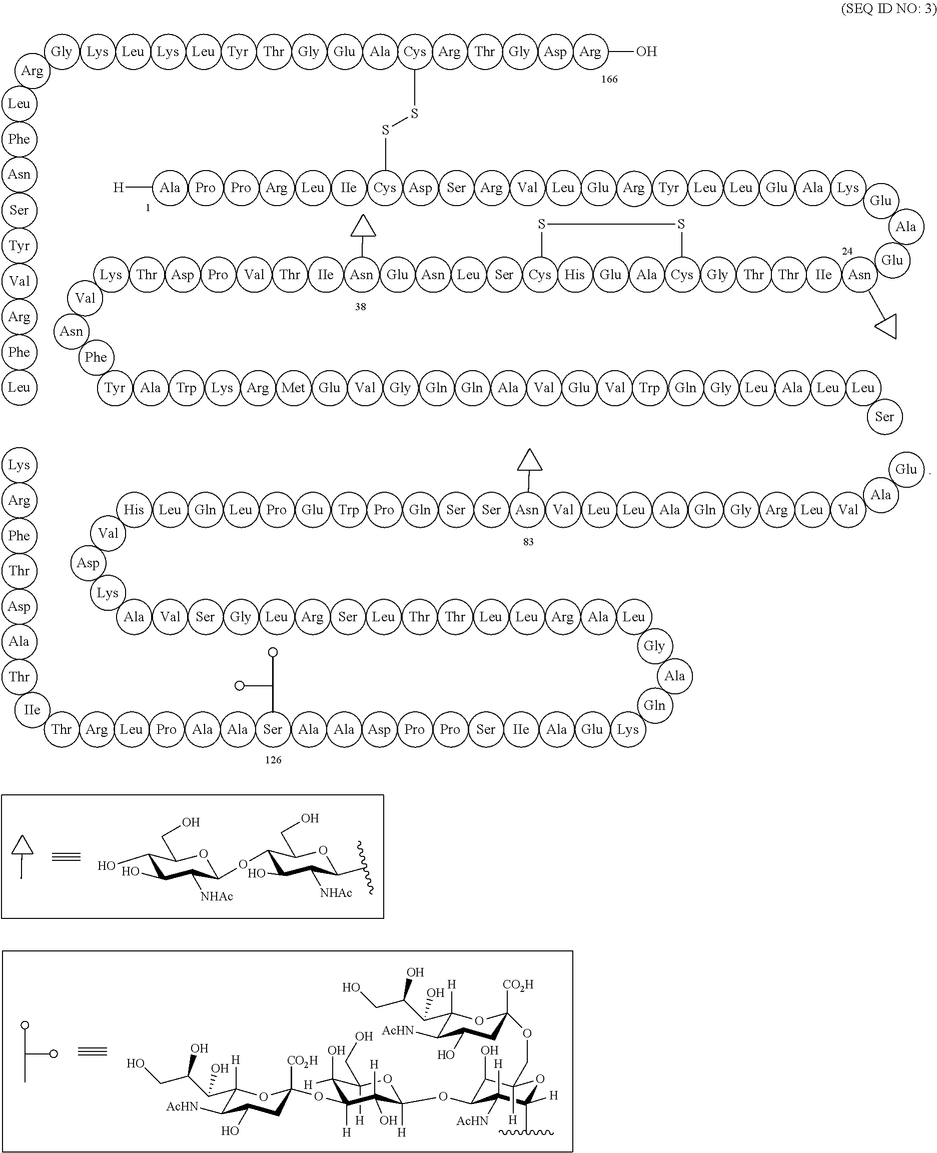

15. The composition of claim 1, wherein the erythropoietin has the following structure:

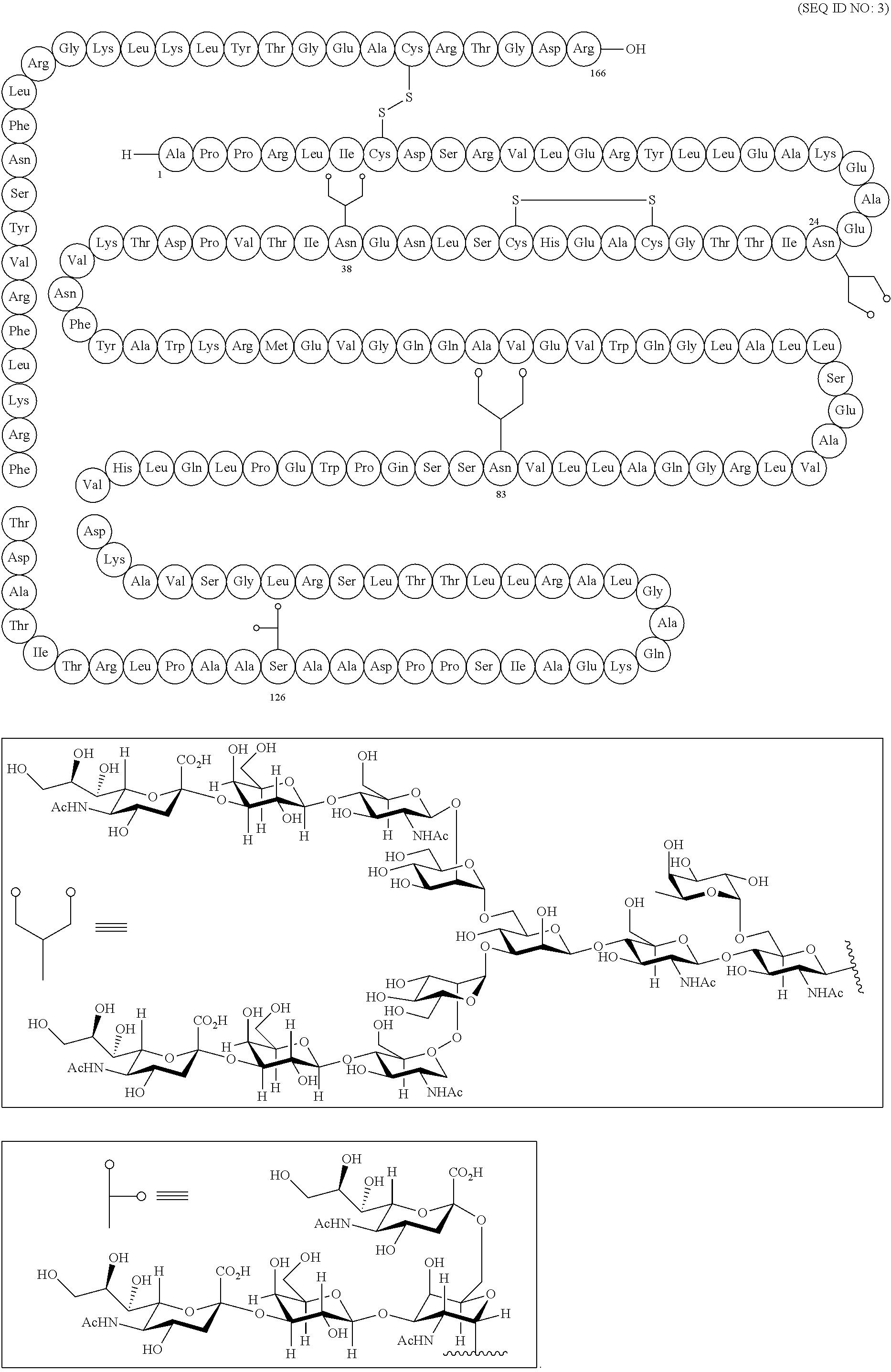

16. A fragment of erythropoietin selected from EPO (1-28), EPO (1-29), EPO (29-78), EPO (30-78), EPO (79-124), EPO (125-166), EPO (128-166), EPO (79-166) and EPO (29-166), wherein the fragment is optionally protected and optionally homogeneously glycosylated.

17. The fragment of claim 16, wherein the fragment is selected from: 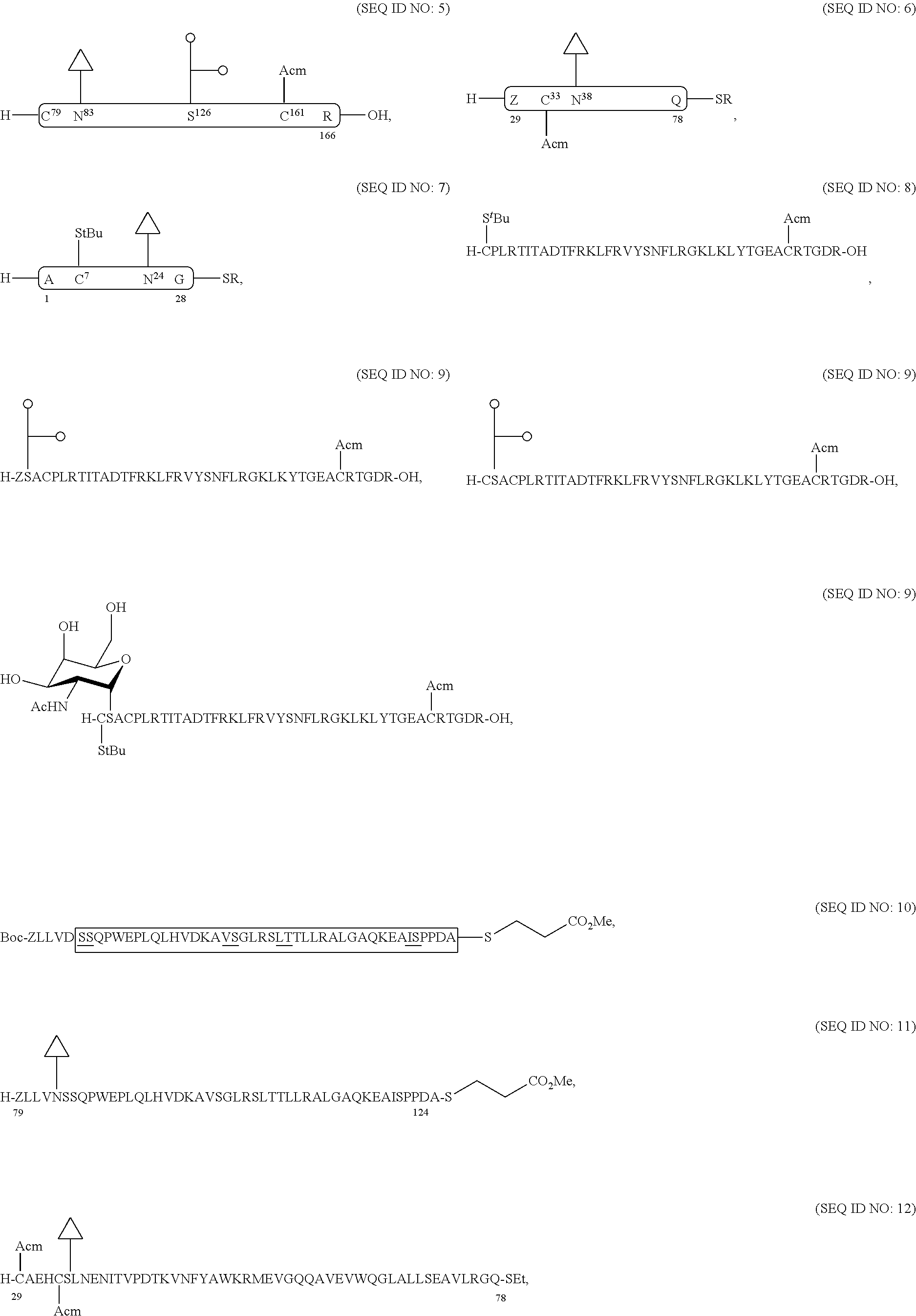 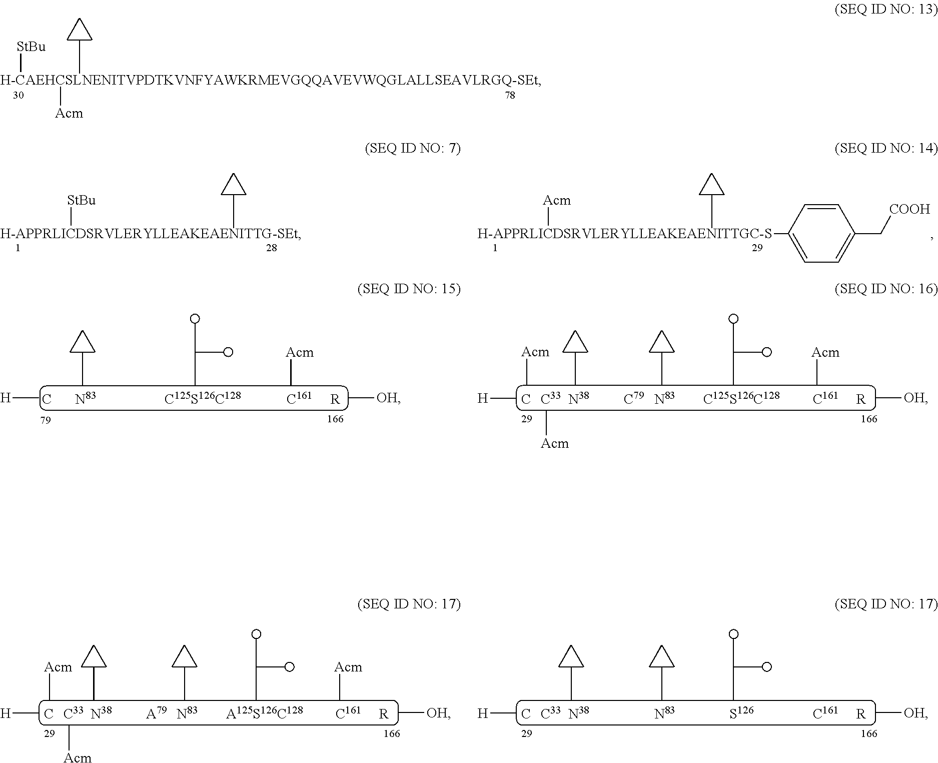
wherein 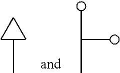
represent different glycans, “Acm” is acetomidomethyl, [symbol] side chain protected sequence, and [symbol] pseudoproline dipeptide.

18. A fragment of claim 16, having 1-10 amino acid substitutions, additions, and/or deletions.

19. A method of preparing a composition of claim 1, comprising the step of ligating one or more EPO fragments.

20. The method of claim 19, wherein the EPO fragments are selected from those of claim 16.

21-26. (canceled)

27. The use of one or more of pseudoproline dipeptides at S⁸⁴S⁸⁵,V⁹⁹S¹⁰⁰, L¹⁰⁵T¹⁰⁶ and I¹¹⁹S¹²⁰ for the synthesis of erythropoietin or its fragments.

28-30. (canceled)

31. A method for studying the structure-function relationships of glycosylated erythropoietin, comprising the use of a composition of claim 1.

32. A method for improving properties of glycosylated erythropoietin, comprising the use of a composition of claim 1.

33. (canceled)
